(19) United States
(12) Patent Application Publication
(10) Pub. No.: US 2017/0044282 A1
(43) Pub. Date: Feb. 16, 2017

(54) TERPENE AND TERPENOID DERIVATIVES CONTAINING VINYL GROUPS FOR THE PREPARATION OF POLYMERS

(71) Applicant: THE UNIVERSITY OF NOTTINGHAM, Nottingham (GB)

(72) Inventors: Steven Howdle, Nottingham (GB); Robert Stockman, Nottingham (GB); Marina Fuentes, Nottingham (GB); Jose Souto, Nottingham (GB); Dominika Regentova, Nottingham (GB); Derek Irvine, Nottingham (GB)

(73) Assignee: The University of Nottingham, Nottingham (GB)

(21) Appl. No.: 15/306,008

(22) PCT Filed: Apr. 22, 2015
(86) PCT No.: PCT/GB2015/051186

§ 371 Date: Oct. 21, 2016

(30) Foreign Application Priority Data
Apr. 22, 2014 (GB) 1407092.4

(51) Int. Cl. C08F 120/68; C07C 29/143; C07C 29/03; C07C 67/14; C07C 69/54

(52) U.S. Cl. CPC C08F 120/68; C07C 67/14; C07C 69/54; C07C 29/03; C07C 29/143; C07C 2102/42; C07C 2101/16; C07C 2101/14

(57) ABSTRACT

The invention relates to a method for producing functionalised monomers, the method comprising: a) providing a starting material selected from terpenes and terpenoids; b) forming a derivative of the starting material by incorporation of a hydroxyl group; c) esterifying the hydroxyl group of the derivative to introduce a moiety containing a vinyl group, so as to produce a functionalised monomer. The functionalised monomer can be polymerised to obtain a bio-derived polymer.

This invention relates to a novel method for the production of bio-derived polymers. Specifically, the invention provides for the production of polymers that are formed from monomers which are derived from raw material that is bio-derived.

### BACKGROUND TO THE INVENTION

Renewable polymers currently capture only a very small share (<5%) of the global polymers market, largely as a result of their perceived high cost and inferior performance compared with synthetic polymers produced from petroleum sources. However, plastics presently account for a global annual usage of more than 7% of fossil fuels and this will undoubtedly grow. Petroleum-derived products will in due course become obsolete and society now demands polymers that are derived from sustainable substitutes.

There is therefore a pressing need for bio-derived feedstocks to replace fossil fuels as the basis for the fine and commodity chemical industries, including the production of polymers. Biorenewable feedstocks are thought of as more environmentally friendly and sustainable by consumers, and can also be biodegradeable in some cases. This leads to manufacturers choosing bio-derived plastics over similar fossil fuel based plastics, provided the cost and quality of the polymer is not significantly different.

In the polymer sector, there is a clear desire for new technologies related to renewable (bio-derived) raw material and biological processes. These have advantages in terms of, for example, stronger industrial competitiveness, raw materials diversification, quicker routes to sustainability, an efficient and sustainable use of natural resources, and a rapid development of new consumer markets.

Any new monomers that are derived from (bio-derived) raw material should be able to be synthesised in an atom efficient manner, and should maximise their renewable component, thus ensuring take-up by industry. However, the polymers as derived from these materials must also offer good or improved performance as compared with synthetic polymers if they are to find a place in the market.

Terpenes and terpenoids are known in the art as naturally occurring materials. They are naturally abundant from plant sources and they are also available as waste products from the wood and citrus juice industries.

Most terpenes have a basic cycloaliphatic structure with an isoprene elementary unit (2-methyl-1,4-butadiene). Terpenoids can be considered as modified terpenes, wherein methyl groups have been moved or removed, or oxygen atoms added.

Terpenes and terpenoids are molecules that arise from biomass, particularly from plants and coniferous trees and are currently available in significant volumes from forestry waste streams. For example, turpentine (˜350 kt per annum) is steadily rising in production volumes. α-pinene and β-pinene account for 45-90% and 1-30% of the total turpentine composition respectively (depending on the kind of tree and geographical area)

D-limonene is currently derived from citrus and coniferous sources for aroma, flavour and pharmaceutical application and is produced at ˜75 kt per annum. Globally, ˜88 Mt per annum of citrus fruit material is used to produce juice and foods (e.g. marmalade) and half of this ends up as citrus waste. While a small portion of this waste is dried and used as animal feed, this is typically not economically viable and a large fraction is simply sent to waste disposal facilities or dumped into the ocean.

Carvone is a naturally occurring terpenoid, being found in both Mentha spicata (spearmint) and Carum carvi (caraway) oils. However, most of the carvone used in industry is synthesised from limonene.

The four most commercially available products from the group of terpenes and terpenoids are:

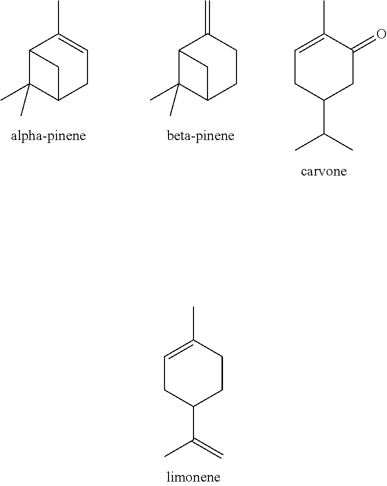

A wide range of methodologies have been applied to polymerise terpenes and have been reviewed extensively. See, for example, M. O. Carmody et al., J. Am. Chem. Soc. 59, 1312 (1937); A. Gandini, Macromolecules 41, 9491 (2008); P. A. Wilbon et al., Macromol. Rapid Commun. 34, 8 (2013); and K. J. Yao et al., Macromolecules 46, 1689 (2013).

The range of polymers that can be formed via cationic polymerisation appears very limited. Amongst the few successes, β-pinene has been shown to polymerise via cationic routes and can form some co-polymers. In order to avoid side reactions, reach high conversions and obtain poly(β-pinene) with high Mn (40,000 g/mol) very low temperatures are required (−40 to −80° C.), however. Thus the conditions required to achieve high yield and good control are not ones that can be easily reproduced on the commercial scale.

Direct polymerisation has in theory seemed a viable opportunity, because many of the terpene and terpenoid structures have unsaturated moieties. However, extensive studies have demonstrated that their reactivities are extremely low and they simply do not polymerise well with free radical initiators. They do not homopolymerise, and even in the presence of a co-monomer (such as styrene or an acrylate) the level of incorporation of β-pinene or d-limonene is minimal and the polymers have only very low molecular weights. See A. Gandini, Macromolecules 41, 9491 (2008).

Recently, ruthenium catalysed ring opening metathesis has been utilised to polymerise just two natural sesquiterpenes, caryophyllene and humulene—see E. Grau et al., Green Chemistry 15, 1112 (2013). The polymers have glass transition temperatures in the range from −15 to −50° C. The methodology cannot, however, be applied to the most readily available terpenes.

Others have taken different approaches and have modified the terpene structures to create more reactive monomers. For example, a Baeyer-Villiger oxidation has been used to create lactone monomers from menthone and carvone; these could then be polymerised via ring-opening using a Zn catalyst to yield new polymers and copolymers with glass transition temperatures in the range of −20 to −27° C. See J. R. Lowe et al., Polymer Chemistry 2, 702 (2011). A key attraction of these materials was that they are, in principle, biodegradable. However their low glass transition temperatures preclude most applications requiring mechanical strength.

In a different approach, careful oxidation of limonene was used to create epoxides that could react with CO₂ to form poly(carbonates) in a reaction catalysed by β-diiminate zinc acetate complexes to yield polymer with controlled molecular weight and a glass transition temperature of around 100° C. See C. M. Byrne et al., J. Am. Chem. Soc. 126, 11404 (2004).

Though promising, both of these examples are limited in the range of polymers that can be achieved and it is difficult to envisage simple commercial routes by which controlled block copolymer structures could be created.

In summary, there is an unmet need for polymers that are obtained from polymerising monomers which are bio-derived materials, especially such polymers that can be obtained via a commercially viable and flexible polymerisation route.

### SUMMARY OF THE INVENTION

A novel method for the production of sustainable, bio-derived polymers has been developed. These polymers are formed from terpenes and terpenoids, which can be obtained from a number of sustainable sources, including tree resin, waste citrus peel or through fermentation. This new technology improves upon the known approaches for making terpene-based polymers, giving products with more desirable properties.

The present invention enables modification of the terpene or terpenoid so as to provide a monomeric unit that can be readily polymerised.

The present invention enables modification of the terpene or terpenoid prior to polymerisation so that the molecular weight and overall structure and morphology of the polymer can be well controlled.

This opens up opportunities for the terpene derived polymer to be used in a variety of applications where existing bio-derived polymers are unsuitable. Two specific example are: plastic cups, where the good thermomechanical stability of the bio-derived polymer is important; and polymer coatings, where the double bonds in the terpenes allow for curing of the coating to enable it to be stabilised. For example, when considering medical applications it is important that coatings as provided on surgical instruments have high thermomechanical stability, so that the instruments can be sterilised at high temperature, e.g. in an autoclave. Clearly, the present invention is not limited to such end uses, but they are examples of where the ability to produce polymers with high Tg values is important.

Meanwhile, the benefits of terpenes and terpenoids as starting materials are retained, in that they are renewable natural feedstocks which are abundant and relatively low cost.

The improved physical properties of the terpene derived polymers can be used to access a wide range of potential end uses across a number of sectors, where existing bio-derived polymers are limited due to their physical properties. These include packaging for food and beverages, textiles, footwear and the automotive industry.

Specifically, the present invention provides:
methods for producing functionalised terpenes or terpenoids, which can be used as monomers;
monomers which are functionalised terpenes or terpenoids;
methods for producing bio-derived polymers by polymerising functionalised terpenes or terpenoids;
bio-derived polymers which are based on repeating units containing functionalised terpenes or terpenoids;
articles comprising bio-derived polymers which are based on repeating units containing functionalised terpenes or terpenoids.

In a first aspect, the invention provides a method for producing functionalised monomers, the method comprising:
a) providing a starting material selected from terpenes and terpenoids;
b) forming a derivative of the starting material by incorporation of a hydroxyl group;
c) esterifying the hydroxyl group of the derivative to introduce a moiety containing a vinyl group, so as to produce a functionalised monomer.

For example, step c) may introduce a —CH═CH₂ group in the form of an acrylate group or derivative thereof (e.g. where one of the vinyl hydrogens is replaced by an alkyl group, such as a methyl group, thus giving a methacrylate group) or in the form of an allyl group, or in the form of a vinyl group.

The functionalised monomer includes a polymerisable functionality, in the form of a —CH═CH₂ group.

In an alternative first aspect, the invention provides a method for producing functionalised monomers, the method comprising:
a) providing a starting material selected from terpenoids, wherein said terpenoid includes a hydroxyl group;
b) optionally forming a derivative of the starting material, provided that said derivative includes a hydroxyl group;
c) esterifying the hydroxyl group to introduce a moiety containing a vinyl group, so as to produce a functionalised monomer.

The functionalised monomer includes a polymerisable functionality, in the form of a —CH═CH₂ group.

In a second aspect, the invention provides a method for producing functionalised monomers, the method comprising:
a) providing a starting material selected from terpenes and terpenoids; and
b) forming a derivative of the starting material by incorporating an organic group including a polymerisable functionality by catalytic introduction of a moiety containing a vinyl group, so as to produce a functionalised monomer.

For example, step b) may introduce a —CH═CH₂ group in the form of an acrylate group or derivative thereof (e.g. where one of the vinyl hydrogens is replaced by an alkyl group, such as a methyl group, thus giving a methacrylate group) or in the form of an allyl group, or in the form of a vinyl group.

The functionalised monomer includes a polymerisable functionality, in the form of a —CH═CH₂ group.

In a third aspect, the invention provides functionalised monomers which are obtained by the method of the first or second aspect.

The functionalised monomers are terpenes or terpenoids that have been modified, wherein the terpene or terpenoid retains its C10 structural unit and the modification comprises the incorporation of an organic group, said organic group including a moiety containing a vinyl group as a polymerisable functionality, and said organic group being linked to the C10 structural unit by an ester linkage.

In one embodiment the organic group provides a —CH═CH₂ group in the form of an acrylate group or derivative thereof (e.g. where one of the vinyl hydrogens is replaced by an alkyl group, such as a methyl group, thus giving a methacrylate group) or in the form of an allyl group, or in the form of a vinyl group.

In one embodiment the modification consists of the incorporation of a single organic group, said organic group including a moiety containing a vinyl group as a polymerisable functionality, and said organic group being linked to the C10 structural unit by an ester linkage.

In a fourth aspect, the invention provides a method for producing bio-derived polymers, the method comprising:
providing functionalised monomers according to the third aspect; and
polymerising said monomers.

In one embodiment the polymerisation is a controlled radical polymerisation. Preferably the polymerisation is by free radical polymerisation in the presence of a chain transfer agent. Other examples of controlled radical polymerisation include CCTP, ATRP and RAFT polymerisation.

However, the invention is not limited to any particular route of polymerisation. For example, the polymerisation could be dispersion polymerisation in supercritical CO₂. Other examples of polymerisation techniques that could be used include homogeneous solution or dispersion, emulsion, suspension, bulk or cell cast polymerisation.

The skilled person will of course be well aware of polymerisation techniques and would be able to select a technique for use.

In a fifth aspect, the invention provides bio-derived polymers which are based on repeating units containing functionalised monomers according to the third aspect.

The bio-derived polymers may be obtained by the method of the fourth aspect.

It may be that the polymers are homopolymers and therefore are based on repeating units containing a single type of functionalised monomers according to the third aspect.

It may be that the polymers are copolymers and therefore are based on repeating units containing two or more monomers. It may be that each monomer type is a functionalised monomer according to the third aspect. Alternatively, it may be that one or more of the monomer types is a functionalised monomer according to the third aspect and one or more of the monomer types is not a functionalised monomer according to the third aspect (e.g. there could be styrene monomers or acrylate monomers or combinations thereof). Thus copolymers can be formed which are based on known monomers already used in the art in combination with monomers according to the invention. The copolymers may, in one embodiment, be block copolymers.

Examples of polymers that can be formed in accordance with the present invention include (but are not limited to) random, statistical, block, alternating, tapered, graft, branched or hyperbranched polymers.

In one embodiment, the bio-derived polymers are crosslinked or crosslinkable. This has benefits in terms of potential end uses. Such polymers may, for example, have improved mechanical strength. They may be useful in end applications involving gels or networks.

The use of the method of the second aspect leads to monomers that include an additional double bond. When these are used as monomers the resulting polymer is crosslinkable.

In addition, regardless of the method of manufacture, the use of a functionalised monomer which is carvone acrylate leads to a polymer which is either crosslinked or crosslinkable.

Carvone acrylate monomer is carvone that has been modified in accordance with the invention.

In this regard, the terpene retains its C10 structural unit and the modification comprises the incorporation of an organic group, said organic group including an acrylate group as a polymerisable functionality, and said organic group being linked to the C10 structural unit via an ester linkage.

Specifically, said organic group is an acrylate group and said acrylate group is linked to the C10 structural unit via its ester linkage.

More specifically, the carvone acrylate monomer is:

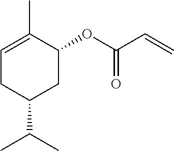

The invention also provides, in a sixth aspect, an article that is (partly or fully) made of polymer, or an article that has a polymer coating (over some, most or all of its surface), wherein the polymer is a bio-derived polymer according to the fifth aspect.

Thus the article may be made from polymer or may contain polymer or may have a polymer coating, wherein the polymer is a bio-derived polymer according to the fifth aspect.

The article may in one embodiment be an article that, in use, may be exposed to high temperatures, such as temperatures of 50° C. or more, such as 70° C. or more, e g. 90° C. or more, or 100° C. or more.

It may be that the article is a polymer coating, wherein said polymer coating provides a heat resistant layer.

It may be that the article is a reusable surgical instrument, wherein the surgical instrument may be sterilised by expose to high temperature, e.g. in an autoclave.

It may be that the article is an article that is used to hold hot liquids, such as a plastic cup.

It may be that the article is an article that is used to hold hot food, such as a microwavable plastic container.

In one aspect, the invention provides a method of forming an article, wherein the method involves forming an article from a polymer, wherein said polymer is a bio-derived polymer according to the fifth aspect and wherein the thus-formed article is (partly or fully) made from said polymer. For example, the article may be molded or extruded from polymer.

In one aspect, the invention provides a method of forming an article, wherein the method involves coating an article with a polymer, wherein said polymer is a bio-derived polymer according to the fifth aspect and wherein a polymer coating is formed over some, most or all of the outer surface of the article.

The key breakthrough is that the polymers produced by the present invention give a range of glass transition temperatures. Therefore the polymers can be tailored dependent on the desired mechanical properties.

To be able to provide bio-derived polymers that have this degree of tailoring is clearly technically advantageous.

### DETAILED DESCRIPTION OF THE INVENTION

The present invention uses terpenes or terpenoids as starting materials. Preferably these are selected from monoterpenes and monoterpenoids.

Terpenes include:

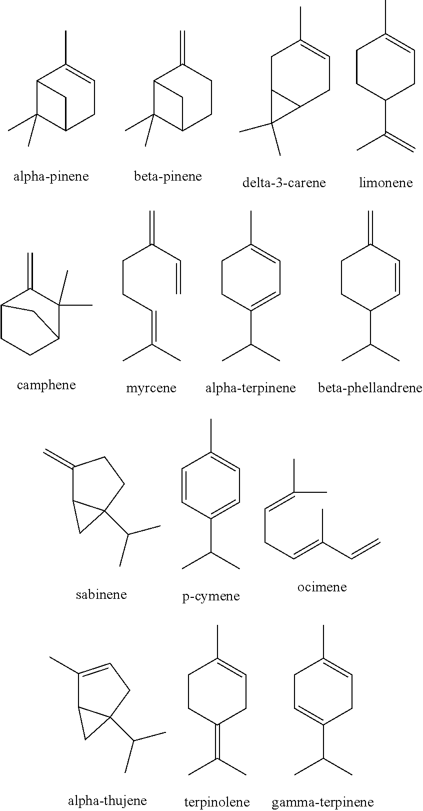

Terpenoids include:

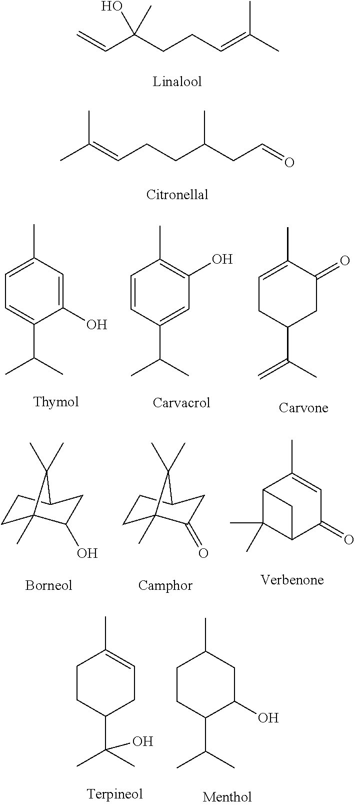

It will be seen that in terpenoids such as carvacrol, borneol, terpineol and menthol a hydroxyl group is present and therefore the alternative first aspect of the invention can be used; there is no requirement to form a derivative that incorporates a hydroxyl group. Of course, optionally, a modification of the terpenoid could still be carried out in step b), as long as the resulting derivative still includes a hydroxyl group.

In one embodiment the terpene or terpenoid used as starting material is a monoterpene or monoterpenoid and is one that has a C10 structure.

This may have an open structure, as in myrcene and ocimene, and in linalool and citronellal, or a closed ring structure, as in pinene, carene, limonene, camphene, terpinene, phellandrene, sabinene, cymene, thujene, and terpinolene, and in thymol, carvacrol, verbenone, carvone, borneol, menthol, camphor and terpineol.

In one embodiment the ring is a six membered closed ring, as in pinene, carene, limonene, camphene, terpinene, phellandrene, cymene, and terpinolene and in thymol, carvacrol, verbenone, carvone, borneol, menthol, camphor and terpineol.

Some of the most commonly available terpenes and terpenoids include:

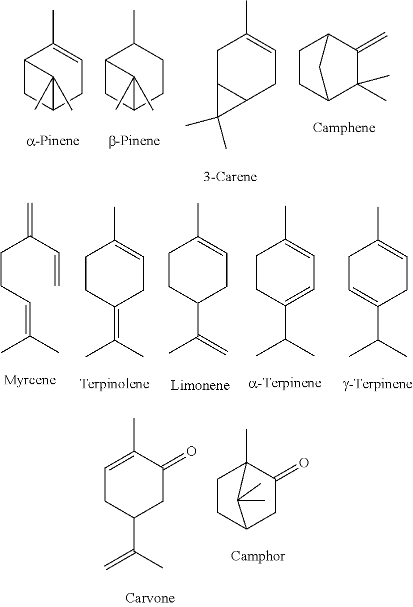

In one embodiment the terpene or terpenoid is one with a general formula of C₁₀H₁₆, e.g. it may be selected from: α-pinene, β-pinene, 3-carene, myrcene, terpinolene, limonene, α-terpinene, γ-terpinene, and camphene, and thymol, carvacrol, verbenone, carvone, camphor, borneol, menthol and terpineol.

In one embodiment the terpene or terpenoid is selected from: α-pinene, β-pinene, 3-carene, terpinolene, limonene, α-terpinene, γ-terpinene, camphene, camphor, carvone, verbenone, menthol and terpineol.

In one embodiment the terpene or terpenoid is selected from: α-pinene, β-pinene, terpinolene, limonene, α-terpinene, γ-terpinene, camphene, camphor, carvone, verbenone, menthol and terpineol.

In one embodiment the terpene or terpenoid is selected from: α-pinene, β-pinene, terpinolene, limonene, camphene, camphor, carvone, verbenone, menthol and terpineol.

In one embodiment the terpene or terpenoid is selected from: α-pinene, β-pinene, limonene, camphene, carvone, verbenone, menthol and terpineol.

In one embodiment the terpene or terpenoid is selected from: α-pinene, β-pinene, camphene, carvone, verbenone, menthol and terpineol.

In general, the larger/more sterically bulky the terpene or terpenoid group, the lower the Tg of the resultant polymer that can be obtained from polymerisation of the monomer. Therefore the terpene or terpenoid group can be selected accordingly, based on the desired Tg of the polymer in light of the intended end use.

As noted above, it may be desired that the polymer has a relatively high Tg, such that articles can be made from the polymer or can be coated with the polymer, where those articles have good thermomechanical stability and so can be used at high temperature. In such embodiments, a terpene or terpenoid group that is relatively compact/not sterically bulky may be selected.

Examples of the starting materials and functionalised monomers that can be produced include:

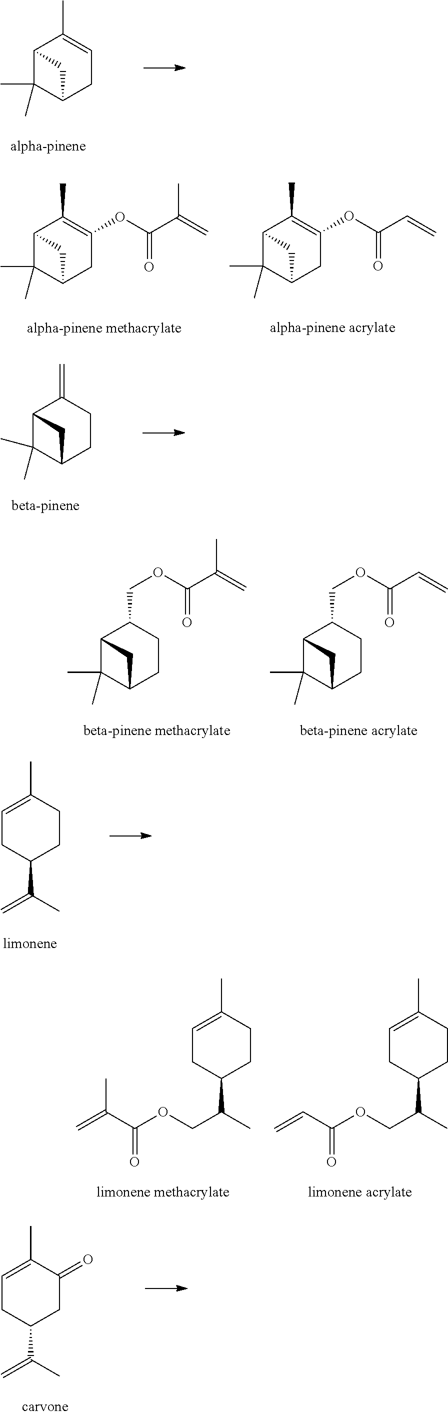

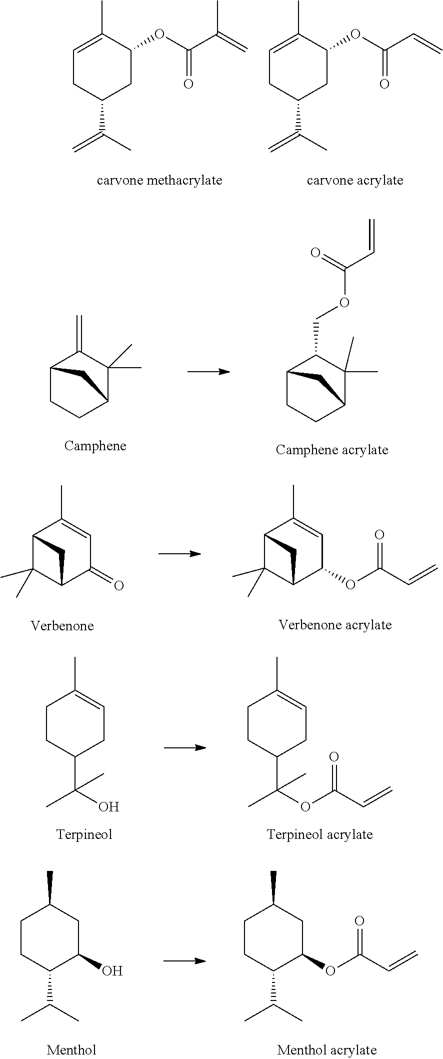

In general, throughout the present document, where structures are illustrated with stereochemistry shown, the skilled reader will appreciate that such a product with the alternate stereochemistry could likewise be used. Thus the invention is not limited to the diastereomers as illustrated but rather all diastereomers are within the scope of the invention.

In the first aspect, the method for producing functionalised monomers comprises:
a) providing a starting material selected from terpenes and terpenoids;
b) forming a derivative of the starting material by incorporation of a hydroxyl group;
c) esterifying the hydroxyl group of the derivative to introduce a moiety containing a vinyl group, so as to produce a functionalised monomer.

For step b), there are two options; the appropriate option depends on whether the starting material has a carbonyl group present.

If the starting material has a carbonyl group present (e.g. it may be one of the terpenoids, such as carvone, verbenone or camphor) then in step b) the carbonyl group is reduced stereoselectively.

This step may be carried out with a reducing agent, e.g. with lithium aluminium hydride or with diisobutylaluminium hydride. Sodium borohydride may also be considered.

The reduction may be carried out in a suitable organic solvent, e.g. THF, MeTHF, diethyl ether, toluene or hexane. The skilled person will appreciate that for reduction with diisobutylaluminium hydride a polar organic solvent such as THF should be used.

The reduction may be carried out at a lowered temperature, e.g. at 10° C. or lower, such as 5° C. or lower, preferably 0° C. or lower, e.g. from 0 to −100° C. or from 0 to −80° C.

The stereoselective reduction may alternatively be carried out using catalytic reduction with H₂, e.g. ruthenium catalysed reduction with H₂. An example of a suitable catalyst is Ru(II)/BINAP.

In general, catalytic hydrogenation of the carbonyl group may be carried out using Ru catalysts or Ir catalysts or chiral oxazoborolidines (CBS catalysts). The ligand may, for example, be a chelating diphosphine or a diamine ligand.

Therefore, for example, the stereoselective reduction could be via the protocol described in T. Okhuma, H. Ikehira, T. Ikariya, R. Noyori, Synlett, 1997, 467. This can stereoselectively reduce carvone, for example, as follows:

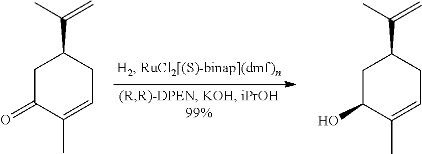

The catalytic reduction may be carried out at room temperature, e.g. from 15 to 25° C.

If the starting material does not have a carbonyl group present (e.g. it may be one of the terpenes, such as one of the pinenes or limonene or terpinolene or camphene), then in step b) the hydroxylation of an alkene group is carried out.

There are various methodologies known in the art that can be used to hydroxylate double bonds, such as the acid catalysed addition of water to a double bond. The invention is not specifically limited to any route.

However, the preferred route is that hydroboration-oxidation of an alkene group is carried out to incorporate a hydroxyl group. This is stereospecific and so is a preferred route. There is no mixture of isomers in the thus-obtained product.

This route therefore involves the use of borane (BH₃) to form an organoborane and then transformation to an alcohol by treatment with basic hydrogen peroxide. This is described in, for example, H. C. Brown, B. S. Subba Rao, J. Am. Chem. Soc. 1959, 81, 6423.

In the hydroboration stage, the borane may be provided in the form of diborane (B₂H₆) or in the form of a hydroborating agent (i.e. in complexed form), e.g. it may be provided as BH3.SMe₂ (BMS), diisoamylborane (Sia₂BH), 9-borabicyclo-[3.3.1]nonane (9-BBN), thexylborane (Thx₂BH), or di(isopropylprenyl)borane (iPP₂BH) or catechol borane. In one embodiment BMS or 9-BBN is used. In another embodiment catechol borane is used.

The borane may be provided in any suitable organic solvent, e.g. THF or MeTHF. In general, ether solvents and hydrocarbon solvents can be considered.

The hydroboration may be carried out at room temperature, e.g. from 15 to 25° C., or a lowered temperature, e.g. at 15° C. or lower, such as 10° C. or lower, preferably 5° C. or lower, e.g. from 5 to −25° C. or from 0 to −10° C.

It may optionally be the case that the hydroboration is catalysed.

In the oxidation stage, the hydrogen peroxide may be used together with any suitable base to provide the required basic conditions. For example, sodium hydroxide may be used or potassium hydroxide may be used.

The treatment with basic hydrogen peroxide is carried out in aqueous solution.

The oxidation step may be carried out at elevated temperature, e.g. at 40° C. or higher, such as 60° C. or higher, preferably 70° C. or higher, e.g. from 70 to 120° C. or from 75 to 100° C.

When considering step c), the skilled person will appreciate that there are various routes available to esterify a hydroxyl group.

For step c), esterification is, in one embodiment, carried out by reaction of the hydroxyl group on the derivative with an acid chloride-containing monomer.

The monomer will be one that contains the —CH═CH₂ group to be introduced.

The acid chloride-containing monomer preferably provides a vinyl, acrylic or allyl group, such that a vinyl, acrylic or allyl ester is formed.

In general, the —CH═CH₂ group to be introduced may be provided in the form of an acrylate group or a derivative thereof (e.g. where one of the vinyl hydrogens is replaced by an alkyl group, such as a methyl group, thus giving a methacrylate group) or in the form of an allyl group, or in the form of a vinyl group.

The acid chloride-containing monomer may, for example, be acryloyl chloride, methacryloyl chloride, or vinylbenzoyl chloride.

In general, the acid chloride-containing monomer is of formula

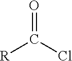

where R is an organic group including a vinyl group as a polymerisable functionality. The organic group R may, for example, be a C2-C18 organic group, especially a C2-C16 or a C2-C14 organic group, such as a C2-C12 organic group.

In one embodiment the organic group R is H₂C═C(X)— where X is hydrogen or C1-C6 alkyl (e.g. C1-C4 alkyl, especially C1, C2 or C3 alkyl), wherein the alkyl group may be straight chain or may be branched. In one embodiment X is hydrogen or methyl, i.e. the acid chloride-containing monomer is acryloyl chloride or methacryloyl chloride.

In general, smaller and less branched the organic group R, the lower the Tg of the resultant polymer that can be obtained from polymerisation of the monomer. Therefore the R group can be selected according, based on the desired Tg of the polymer in light of the intended end use. For example, acrylate leads to a lower Tg than methacrylate.

The esterification is suitably carried out under basic conditions. The esterification may therefore be carried out in the presence of a base, for example trimethylamine or pyridine.

The esterification may be carried out in a suitable organic solvent, e.g. THF, MeTHF, dichloromethane, diethyl ether, toluene or hexane.

Methyltetrahydrofuran (MeTHF) may be preferred as it is benign and is seen as “green” but still gives good isolated yield and stereoselectivity.

The esterification may be carried out at room temperature, e.g. from 15 to 25° C., or at a lowered temperature, e.g. at 15° C. or lower, such as 10° C. or lower, preferably 5° C. or lower, e.g. from 5 to −25° C. or from 0 to −10° C.

In an alternative first aspect, the invention provides a method for producing functionalised monomers, the method comprising:
a) providing a starting material selected from terpenoids, wherein said terpenoid includes a hydroxyl group;
b) optionally forming a derivative of the starting material, provided that said derivative includes a hydroxyl group;
c) esterifying the hydroxyl group to introduce a moiety containing a vinyl group, so as to produce a functionalised monomer.

This alternative first aspect is utilised when the starting material is a terpenoid that already includes a hydroxyl group, such as carvacrol, borneol, terpineol or menthol.

It will be appreciated that step b) then becomes optional. There is no need to further derivatise the starting material. Indeed preferably, step b) is not carried out and the terpenoid that already includes a hydroxyl group is used directly in step c). However, it will be appreciated that as long as the material has a hydroxyl group when step c) is effected, in theory the skilled person could choose to modify the terpenoid starting material—and this is not precluded from the invention.

Step c) is as described above.

In both the first aspect and the alternative first aspect the method is advantageous in that it is simple but robust chemistry. It can be used to synthesise new monomers on a scale such that they can be polymerised.

It is, however, recognised that from the atom economy point of view, this strategy is not “green”. The main by-products of this synthetic route are boron, lithium and aluminium salts coming from step b) and ammonium chloride that precipitates during step c). There may therefore be a desire for a “greener” route to the monomers.

In one embodiment, in both the first aspect and the alternative first aspect, the method of the first aspect is made more “green” by avoiding generating chloride-containing waste products in step c).

For step c), esterification is, therefore in one embodiment, carried out with a carboxylic acid-containing monomer in the presence of a coupling agent.

The monomer will be one that contains the —CH═CH₂ group to be introduced.

The carboxylic acid-containing monomer preferably provides a vinyl, acrylic or allyl group, such that a vinyl, acrylic or allyl ester is formed.

In general, the —CH═CH₂ group to be introduced may be provided in the form of an acrylate group or derivative thereof (e.g. where one of the vinyl hydrogens is replaced by an alkyl group, such as a methyl group, thus giving a methacrylate group) or in the form of an allyl group, or in the form of a vinyl group.

The carboxylic acid-containing monomer may, for example, be acrylic acid, methacrylic acid, or vinylbenzoic acid.

In general, the carboxylic acid-containing monomer is of formula

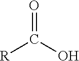

where R is an organic group including a vinyl group as a polymerisable functionality. The organic group R may, for example, be a C2-C18 organic group, especially a C2-C16 or a C2-C14 organic group, such as a C2-C12 organic group.

In one embodiment the organic group R is H₂C═C(X)— where X is hydrogen or C1-C6 alkyl (e.g. C1-C4 alkyl, especially C1, C2 or C3 alkyl), wherein the alkyl group may be straight chain or may be branched. In one embodiment X is hydrogen or methyl, i.e. the carboxylic acid-containing monomer is acrylic acid or methacrylic acid.

A benefit of using acrylic acid or methacrylic acid or the like as the carboxylic acid-containing monomer is that they are easily accessible from nature. This therefore allows the method to be more “bio-derived”. There can be a demand for products to have as high a proportion of “bio-derived”/naturally sourced starting materials as possible.

The coupling agent may be any coupling agent able to couple a carboxylic acid with an alcohol. One suitable coupling agent is propylphosphonic anhydride (known as T3P®). Alternative coupling agents include bis(2-oxo-3-oxazolidinyl)phosphonic chloride (BOP-C1), norborn-5-ene-2,3-dicarboximido diphenyl phosphate (NDPP), pentafluorophenyl diphenylphosphinate (FDPP) and diethyl phosphorocyanidate (DEPC).

The reaction using propylphosphonic anhydride with acrylic acid is:

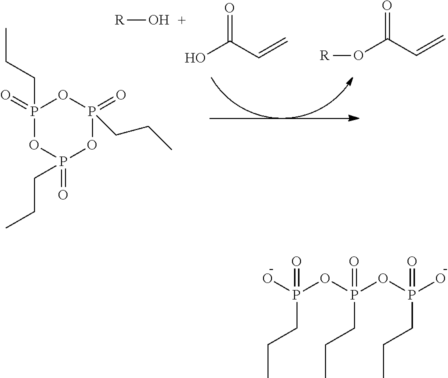

A benefit of using propylphosphonic anhydride is that it can be used at room temperature. Further, the resulting T3P® by-product is easily removed by aqueous work-up, e.g. two washes with water. No further purification is necessary. It is seen as a “green” reagent and is non-hazardous.

The esterification is optionally carried out under basic conditions. The esterification may therefore be carried out in the presence of a base, for example triethylamine or pyridine.

The esterification may be carried out in a suitable organic solvent, e.g. THF, MeTHF, dichloromethane, ethyl acetate, diethyl ether, toluene or hexane.

Methyltetrahydrofuran (MeTHF) may be preferred as it is benign and is seen as “green” but still gives good isolated yield and stereoselectivity.

The esterification may be carried out at room temperature, e.g. from 15 to 25° C., or at a lowered temperature, e.g. at 15° C. or lower, such as 10° C. or lower, or 5° C. or lower, e.g. from 5 to −25° C. or from 0 to −10° C.

This method is advantageous in that it is simple but robust chemistry. It can be used to synthesise new monomers on a scale such that they can be polymerised. In addition, this route allows the monomers generated to be highly bio-derived, e.g. as much as 92% bio-derived.

It is, however, recognised that from the atom economy point of view, this strategy is not “green”. There are still by-products of this synthetic route coming from step b), namely boron, lithium and aluminium salt (except in the alternate first aspect where step b) is not required). In addition, step c) does generate waste. There may therefore be a desire for a yet “greener” route to the monomers.

Accordingly, an alternate method has also been developed. This uses metal complexes, such as palladium complexes, as catalysts and permits a one pot reaction.

In a second aspect, therefore, the invention provides a method for producing functionalised monomers, the method comprising:
a) providing a starting material selected from terpenes and terpenoids; and
b) forming a derivative of the starting material by incorporating an organic group including a polymerisable functionality by catalytic introduction of a moiety containing a vinyl group, so as to produce a functionalised monomer.

For example, step b) may introduce a —CH═CH₂ group in the form of an acrylate group or a derivative thereof (e.g. where one of the vinyl hydrogens is replaced by an alkyl group, such as a methyl group, thus giving a methacrylate group) or in the form of an allyl group, or in the form of a vinyl group.

For step b), the catalytic reaction is carried out with a carboxylic acid-containing monomer in the presence of a catalyst.

The carboxylic acid-containing monomer preferably provides a vinyl, acrylic or allyl group, such that a vinyl, acrylic or allyl ester is formed.

In general, the —CH═CH₂ group to be introduced may be provided in the form of an acrylate group or derivative thereof (e.g. where one of the vinyl hydrogens is replaced by an alkyl group, such as a methyl group, thus giving a methacrylate group) or in the form of an allyl group, or in the form of a vinyl group.

The carboxylic acid-containing monomer may, for example, be acrylic acid, methacrylic acid, or vinylbenzoic acid.

In general, the carboxylic acid-containing monomer is of formula

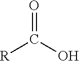

where R is an organic group including vinyl group as a polymerisable functionality. The organic group R may, for example, be a C2-C18 organic group, especially a C2-C16 or a C2-C14 organic group, such as a C2-C12 organic group.

In one embodiment the organic group R is H₂C═C(X)— where X is hydrogen or C1-C6 alkyl (e.g. C1-C4 alkyl, especially C1, C2 or C3 alkyl), wherein the alkyl group may be straight chain or may be branched. In one embodiment X is hydrogen or methyl, i.e. the carboxylic acid-containing monomer is acrylic acid or methacrylic acid.

The catalyst may be any metal catalyst that is known for acetylation or for similar catalytic reactions, such as allylic esterification or allylic acetoxylation.

In one embodiment, it is a Pd(II)-catalyst. Pd(II)-catalysts are known in the art for use in both industrial and academic synthetic chemistry laboratories as a powerful methodology for the formation of C—C and C-heteroatom bonds.

The catalyst may, for example, be Pd₂(dba)₃ or Pd(OAc)₂ or Pd(PPh₃)₄ or Pd(P^{t}Bu₃)₂.

In another embodiment it is a Fe(III)-catalyst.

The catalyst may suitably be Pd(OAc)₂.

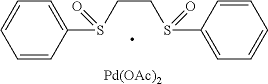 [SMILES: O=S(CCS(=O)c1ccccc1)c1ccccc1 ; "Pd(OAc)2"]

The use of a Pd catalyst in the second aspect yields analogous monomers to those obtained with the methods of the first aspect, but with an additional double bond present.

Due to the presence of the additional double bond, they are crosslinkable. This is therefore technically beneficial.

The catalytic reaction is suitably carried out in the presence of a re-oxidation agent to regenerate the catalyst. This may, for example, be 1,4-benzoquinone. 1,4-benzoquinone also acts to inhibit polymerisation of the carboxylic acid-containing monomer, e.g. acrylic acid.

In general, any stoichiometric oxidant, such as CuCl₂, Cu(OAc)₂, tert-butyl hydroperoxide (TBHP), MnO₂, or HNO₃ could be used to regenerate the catalyst.

An alternative to benzoquinone that has been used in allylic C—H acetoxylation is 4,5-diazafluorenone and this could therefore be considered for use.

In general, any known reagent that regenerates the chosen catalyst may be contemplated. The catalysts are not new per se and therefore their regeneration is known in the art, e.g. in the context of their use as acetylation catalysts or for similar catalytic reactions, such as allylic esterification or allylic acetoxylation.

It clearly is desirable that the reagent used does not generate radicals so that it does not result in the monomer that is being produced undergoing radical initiated polymerisation before it has been isolated.

In general, for all the methods of the invention that are described in the first and second aspects, it is preferred that no reagents are present that generate radicals, such that the monomer that is being produced does not undergo radical initiated polymerisation before it has been isolated.

The catalytic reaction is optionally carried out under basic conditions. The esterification may therefore be carried out in the presence of a base, for example trimethylamine or pyridine.

The catalytic reaction does not require the presence of a separate solvent. This is a further advantage of this method of the invention. It has been identified that the carboxylic acid-containing monomer, e.g. acrylic acid or methacrylic acid, may serve as the solvent as well as the reagent. This is clearly beneficial from a “green” perspective, as it reduces waste and increases the proportion of the agents used in the method that may be naturally sourced. As noted above, both acrylic acid and methacrylic acid are easily accessible from nature. The use of such agents as reactant and solvent therefore allows the method to be more “bio-derived”.

However, the reaction may, alternatively, be carried out in a suitable organic solvent, e.g. THF, MeTHF, ethyl acetate, diethyl ether, toluene or hexane.

The esterification may be carried out at room temperature, e.g. from 15 to 25° C., or at a lowered temperature, e.g. at 15° C. or lower, such as 10° C. or lower, or 5° C. or lower, e.g. from 5 to −25° C. or from 0 to −10° C.

This method is advantageous from an atom economy point of view, and may be considered “green”.

The invention provides, in a third aspect, functionalised monomers which are obtainable by the method of the first or second aspect.

It may be that the monomers have been obtained by the method of the first or second aspect.

The functionalised monomers are terpenes or terpenoids that have been modified, wherein the terpene or terpenoid retains its C10 structural unit and the modification comprises the incorporation of an organic group, said organic group including a moiety containing a vinyl group as a polymerisable functionality, and said organic group being linked to the C10 structural unit by an ester linkage.

In one embodiment the organic group provides a —CH═CH₂ group in the form of an acrylate group or derivative thereof (e.g. where one of the vinyl hydrogens is replaced by an alkyl group, such as a methyl group, thus giving a methacrylate group) or in the form of an allyl group, or in the form of a vinyl group.

In one embodiment the modification consists of the incorporation of a single organic group, said organic group including a moiety containing a vinyl group as a polymerisable functionality, and said organic group being linked to the C10 structural unit by an ester linkage.

The organic group may, for example, be a C2-C18 organic group, especially a C2-C16 or a C2-C14 organic group, such as a C2-C12 organic group.

In one embodiment the organic group is H₂C═C(X)— where X is hydrogen or C1-C6 alkyl (e.g. C1-C4 alkyl, especially C1, C2 or C3 alkyl), wherein the alkyl group may be straight chain or may be branched. In one embodiment X is hydrogen or methyl.

In one embodiment the monomer is a terpene or terpenoid that has been modified, wherein the terpene or terpenoid retains its C10 structural unit and the modification comprises the incorporation of a C1-C6 organic group, said organic group including a moiety containing a vinyl group as a polymerisable functionality, and said organic group being linked to the C10 structural unit by an ester linkage.

It may be that the organic group is a C1-05 organic group containing a vinyl group as a polymerisable functionality; for example the organic group may be a C1-C4 organic group containing a vinyl group as polymerisable functionality, such as a C1, C2 or C3 organic group containing a vinyl group as a polymerisable functionality.

In one embodiment the functionalised monomers are terpenes or terpenoids that have been modified, wherein the terpene or terpenoid retains its C10 structural unit and wherein the modification comprises the incorporation of an acrylate or methacrylate group. Possible terpenes and terpenoids are discussed above.

In one embodiment the functionalised monomers are terpenes or terpenoids that have been modified, wherein the terpene or terpenoid retains its C10 structural unit and wherein the modification comprises the incorporation of an acrylate or methacrylate group, and wherein the terpene or terpenoid is selected from: α-pinene, β-pinene, terpinolene, limonene, α-terpinene, γ-terpinene, camphene, camphor, carvone, verbenone, menthol and terpineol. It may be that the terpene or terpenoid is selected from: α-pinene, β-pinene, terpinolene, limonene, camphene, camphor, carvone, verbenone, menthol and terpineol. For example, the terpene or terpenoid may be selected from: α-pinene, β-pinene, camphene, carvone, verbenone, menthol and terpineol.

In one embodiment, the functionalised monomer has been made by the method of the second aspect, and thus the product contains an additional double bond as compared to the starting terpene or terpenoid.

Such monomers are particularly beneficial, because the additional double bond makes the resultant polymers crosslinkable. As the skilled person will appreciate, being able to crosslink a polymer means that the properties of that polymer can be improved or changed.

In particular, the introduction of cross linking bonds between adjacent molecular chains adds stability at higher temperatures. Crosslinked molecular chains are much more resistant to flow when stress is applied.

The introduction of cross linking bonds between adjacent molecular chains also makes the product suitable for applications that involve gels or networks.

There may, for example, be higher tensile strength, improved resistance to stress cracking, and improved mechanical properties at high temperature in a crosslinked polymer.

In one embodiment, the functionalised monomer is selected from the group consisting of:

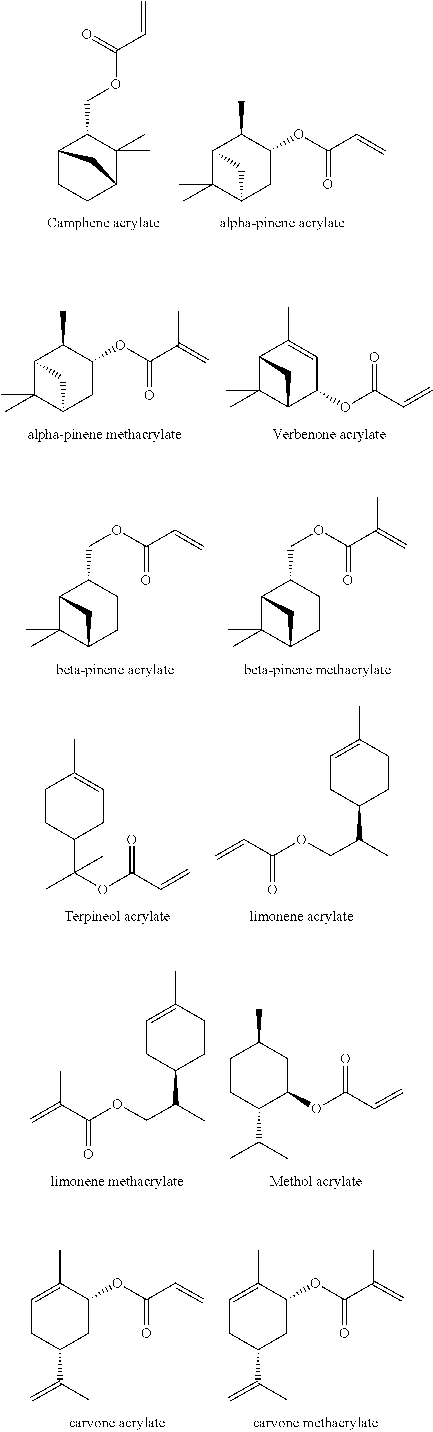

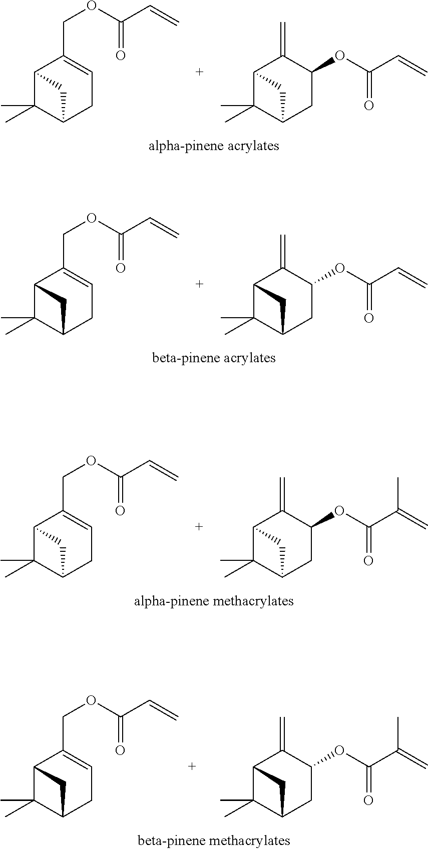

In one embodiment, the functionalised monomer is carvone acrylate. As noted above, this is a particularly beneficial monomer because on polymerisation it forms a crosslinkable or crosslinked polymer. This is the case regardless of whether the product is made by the method of the first aspect or the second aspect.

In a fourth aspect, the invention provides a method for producing bio-derived polymers, the method comprising:
providing functionalised monomers according to the third aspect; and
polymerising said monomers.

In one embodiment the step of providing functionalised monomers according to the third aspect comprises carrying out the method of the first aspect or carrying out the method of the second aspect.

In one embodiment the polymerisation is a controlled radical polymerisation technique. However the invention is not limited to any particular polymerisation technique.

Preferably the polymerisation is by free radical polymerisation in the presence of a chain transfer agent.

Other examples of controlled radical polymerisation includes CCTP, ATRP and RAFT polymerisation.

In another embodiment the polymerisation could be dispersion polymerisation in supercritical CO₂. Other examples of polymerisation techniques that could be used include homogeneous solution or dispersion, emulsion, suspension, bulk or cell cast polymerisation.

It may be that the polymers are homopolymers and therefore the polymerisation is based on a single type of functionalised monomers according to the third aspect.

It may, alternatively, be that the polymers are copolymers and the polymerisation is based on two or more different monomer types, each of which is a functionalised monomer according to the third aspect.

It may, alternatively, be that the polymers are copolymers and the polymerisation is based on two or more different monomer types, one or more of which is a functionalised monomer according to the third aspect and one or more of which is not a functionalised monomer according to the third aspect (e.g. these could be styrene monomers or acrylate monomers or combinations thereof).

The copolymers as formed may, in one embodiment, be block copolymers.

Examples of polymers that can be formed in accordance with the present invention include (but are not limited to) random, statistical, block, alternating, tapered, graft, branched or hyperbranched polymers.

The polymerisation can be carried out using any known polymerisation technique. It will be appreciated that the presence of the vinyl group in the monomers opens up the possibility for straightforward polymerisation by a range of polymerisation techniques.

In one embodiment the polymerisation is a controlled radical polymerisation. Preferably the polymerisation is by free radical polymerisation in the presence of a chain transfer agent. Other examples of controlled radical polymerisation includes CCTP, ATRP and RAFT polymerisation.

However, the invention is not limited to any particular route of polymerisation. For example, the polymerisation could be dispersion polymerisation in supercritical CO₂. Other examples of polymerisation techniques that could be used include homogeneous solution or dispersion, emulsion, suspension, bulk or cell cast polymerisation.

In one embodiment, the polymerisation is effected by chain transfer polymerisation.

The polymerisation can be carried out using any suitable chain transfer agent.

In one embodiment, the chain transfer agent (CTA) is a thiol, such as dodecyl mercaptan (DDM).

In another embodiment, the chain transfer agent is a terpene. For example, it may be terpinolene or gamma-terpinene. The use of such terpenes as chain transfer agents is known in the art. These chain transfer agent terpenes are discussed further below. As terpenes are natural materials the use of a terpene chain transfer agent may be preferred to make the process more “green”.

As the skilled person will appreciate, the Tg of the polymer is affected by the molecular weight of the polymer. The molecular weight of the polymer will depend on the concentration of chain transfer agent (regardless of whether it is a terpene or a conventional CTA like DDM), with a higher concentration of CTA leading to a lower molecular weight.

In general, therefore, the use of a larger amount of chain transfer agent leads to a lower Tg for the resultant polymer that can be obtained from polymerisation of the monomer. Therefore the amount of chain transfer agent can be selected accordingly, based on the desired Tg of the polymer in light of the intended end use.

In a fifth aspect, the invention provides bio-derived polymers which are based on repeating units containing functionalised monomers according to the third aspect.

The bio-derived polymers may be obtainable by the method of the fourth aspect.

The bio-derived polymers may have been obtained by using the method of the fourth aspect.

The bio-derived polymers may be homopolymers, in which case they are formed from repeating units containing a single type of the functionalised monomers according to the third aspect.

The bio-derived polymers may be co-polymers formed from repeating units containing two or more types of the functionalised monomers according to the third aspect.

The bio-derived polymers may be co-polymers formed from repeating units containing one or more types of the functionalised monomers according to the third aspect and one or more monomers that are not functionalised monomers according to the third aspect. These monomers that are not according to the invention may be any known monomer type, e.g. styrenes or acrylates.

In one embodiment, the polymers are homopolymers or co-polymers based on repeating units containing functionalised monomers, wherein the functionalised monomers are terpenes or terpenoids that have been modified, wherein the terpene or terpenoid retains its C10 structural unit and wherein the modification comprises the incorporation of an acrylate or methacrylate group, and wherein the terpene or terpenoid is selected from: α-pinene, β-pinene, terpinolene, limonene, α-terpinene, γ-terpinene, camphene, camphor, carvone, verbenone, menthol and terpineol. It may be that the terpene or terpenoid is selected from: α-pinene, β-pinene, terpinolene, limonene, camphene, camphor, carvone, verbenone, menthol and terpineol. For example, the terpene or terpenoid may be selected from: α-pinene, β-pinene, camphene, carvone, verbenone, menthol and terpineol.

In one embodiment, the polymers are homopolymers or co-polymers based on repeating units containing functionalised monomers, wherein the functionalised monomers are selected from the group consisting of:

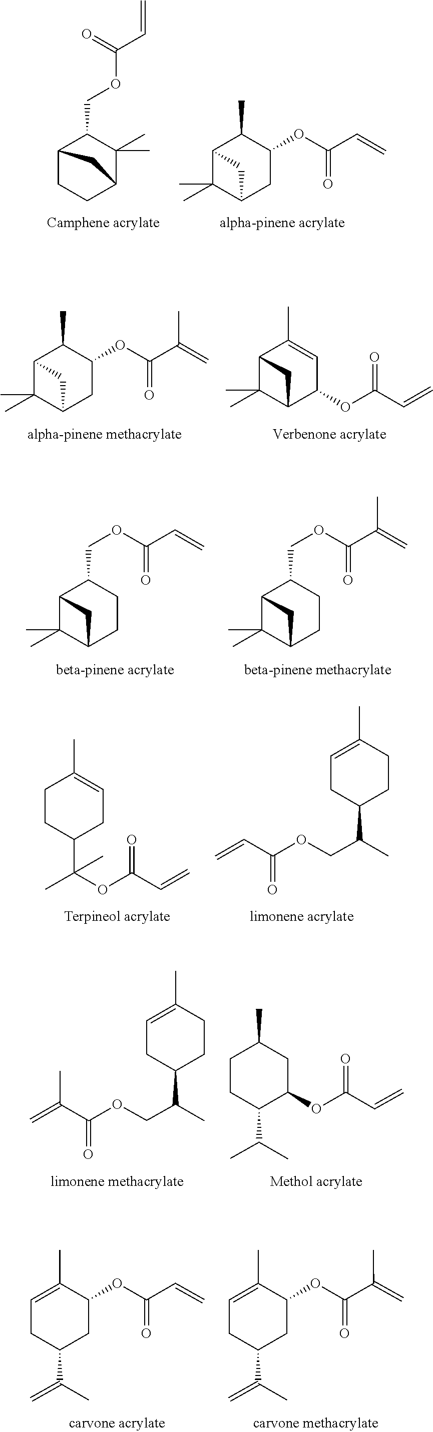

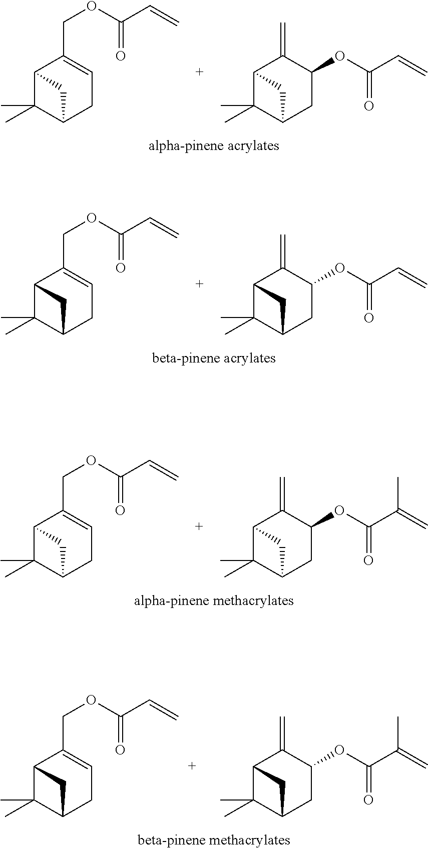

In one embodiment, the functionalised monomer comprises carvone acrylate. Thus the polymer may be a homopolymer of carvone acrylate or a co-polymer where one of the monomers is carvone acrylate. In the latter case, preferably carvone acrylate makes up 50% or more by weight of the monomer repeating units, such as 60% or more or 70% or more, preferably 80% or more, e.g. 90% or more or 95% or more. It may be that carvone acrylate makes up from 50% to 99% by weight of the monomer repeating units, such as from 60% to 95% by weight.

As noted above, this is a particularly beneficial monomer because on polymerisation it forms a crosslinkable or crosslinked polymer.

In one embodiment, the functionalised monomer comprises monomer as obtainable by (or as obtained by) the method of the second aspect. The polymer may be a homopolymer or may be a co-polymer where one of the monomers is monomer as obtainable by (or as obtained by) the method of the second aspect. In the latter case, preferably said monomer as obtainable by (or as obtained by) the method of the second aspect makes up 50% or more by weight of the monomer repeating units, such as 60% or more or 70% or more, preferably 80% or more, e.g. 90% or more or 95% or more. It may be that monomer as obtainable by (or as obtained by) the method of the second aspect makes up from 50% to 99% by weight of the monomer repeating units, such as from 60% to 95% by weight.

As noted above, this is a particularly beneficial monomer because on polymerisation it forms a crosslinkable or crosslinked polymer.

In one such embodiment the functionalised monomer is an acrylate or methacrylate derivative of a terpene or terpenoid, said monomer being obtainable by (or obtained by) the method of the second aspect.

The invention also provides, in a sixth aspect, an article made of polymer or an article that contains polymer or an article that has a polymer coating, wherein the polymer is a bio-derived polymer according to the fifth aspect.

The article may in one embodiment be an article that, in use, may be exposed to high temperatures, such as temperatures of 50° C. or more, such as 70° C. or more, e g. 90° C. or more, or 100° C. or more.

It may be that the article is a polymer coating, wherein said polymer coating provides a heat resistant layer.

It may be that the article is a reusable surgical instrument, wherein the surgical instrument may be sterilised by expose to high temperature, e.g. in an autoclave.

It may be that the article is an article that is used to hold hot liquids, such as a plastic cup.

It may be that the article is an article that is used to hold hot food, such as a microwavable plastic container.

However, the invention is not limited to certain types of plastic articles or plastic coated articles.

It will be appreciated that the articles of the invention can be made by conventional methods, e.g. by molding or extruding the polymer into the desired shape or by coating some, most or all of the surface of a pre-formed product with the polymer.

In one embodiment, the method of the fourth aspect is carried out and then the thus-formed polymer of used to form some or all of an article (e.g. by molding or extrusion) or is used to coat a product to provide a coated article.

Initial Discussion of the Experimental Work and the Conclusions that can be Drawn

The four most commercially available terpenes (limonene, carvone, and α- and β-pinene) were transformed following hydroboration-oxidation tandem protocol to install a hydroxyl functionality. In the case of carvone, an alcohol was formed by reduction of the enone functionality with lithium aluminium hydride

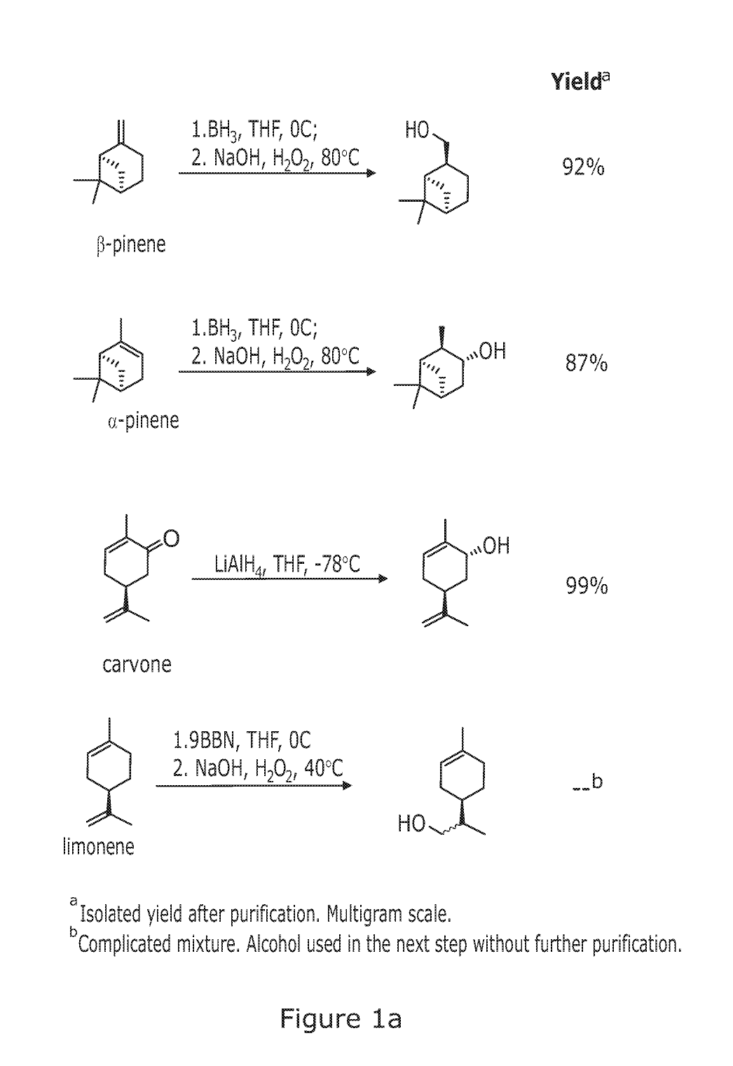

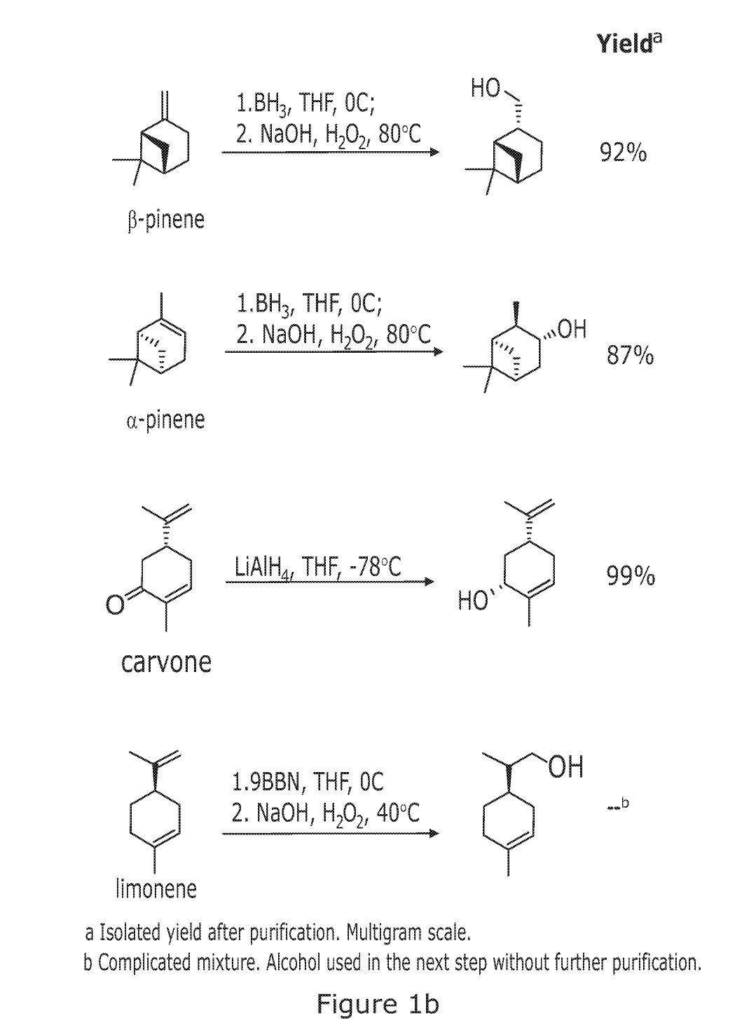

See FIG. 1a for the reaction schemes and yields. See FIG. 1b for a version of this table that reflects the inventors' current understanding of the structures obtained.

For a comprehensive discussion of the hydroboration/oxidation of limonene, see: A. F. Thomas and Y. Bessiere, Nat. Prod. Rep., 1989, 291.

The alcohol derivatives are obtained as pure enantiomers in every case except for the limonene derivative, which is obtained as a 1:1 mixture of diastereomers, together with other impurities.

Once isolated (except limonene, which was used in the next step without further purification), the hydroxy terpenoids were esterified by treatment with acryloyl or methacryloyl chloride and Et3N as a base, yielding the corresponding acrylate and methacrylate derivatives.

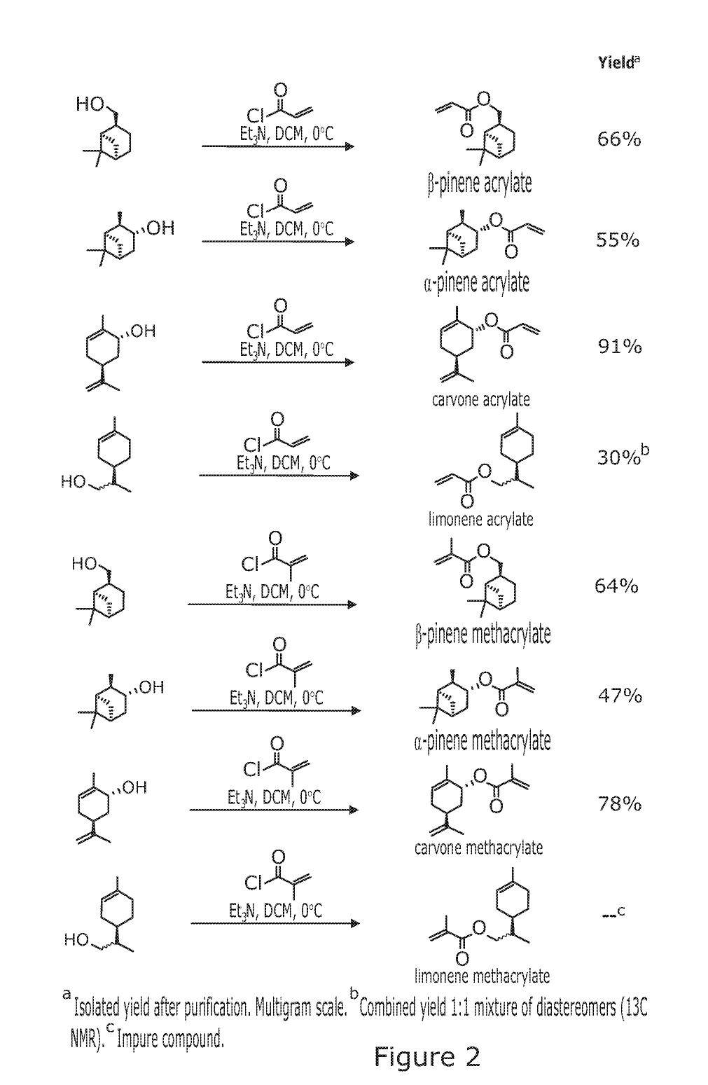

See FIG. 2 for the reaction schemes and yields.

This chemistry is robust, with main by-products being boric acid and hydrochloric acid. Whilst this synthetic approach is not “green”, and likely is not appropriate for the commercial scale, our approach has allowed the simple synthesis of the monomers at such a scale that they can then be polymerised.

In FIG. 2, the absolute configurations of the enantiomers are shown. All of the new monomers, except the limonene derivatives, are enantiomerically pure. The limonene methacrylate derivative was obtained together with an alkene-containing impurity that co-eluted in the chromatographic column. Therefore, the synthesis of this compound needs to be optimised.

In this regard, limonene methacrylate is obtained together with an unsaturated impurity (detected by NMR) that shows the same retention factor as the monomer in chromatographic columns. Therefore this impurity coelutes with the product. Thus, an alternative method is required. The most common procedure to purify the liquid (meth)acrylate monomers, as practised in industry, is by fractional distillation under reduced pressure. This can readily be achieved

In this regard, the major impurity is menth-1-en-9-al, which has a boiling point of 82-85° C. at 8 mmHg (Ref: Meinwald, J.; Jones, T. H., J. Am. Chem. Soc., 1978, 100, 1883-1886). Thus the impurity can be easily removed by reduced pressure distillation.

In order to avoid polymerisation reactions a radical inhibitor such as phenothiazine (PTZ) should be added to the mixture.

Relevant teaching in this regard can be found in:
Gunes, D.; Karagoz, B.; Bicak, N., Synthesis of methacrylate-based functional monomers via boron ester acidolysis and their polymerization. Des. Monomers Polym. 2009, 12 (5), 445-454.
U.S. Pat. No. 2,341,282
Mosnacek, J.; Nicolay, R.; Kar, K. K.; Fruchey, S. O.; Cloeter, M. D.; Hamer, R. S.; Matyjaszewski, K., Efficient Polymerization Inhibition Systems for Acrylic Acid Distillation: New Liquid-Phase Inhibitors. Ind. Eng. Chem. Res. 2012, 51(10), 3910-3915.
JP2002/275125A.

These new terpene-based acrylate and methacrylate derivatives have been polymerized to form new terpene-based polymers, using standard free radical strategies, e.g.:

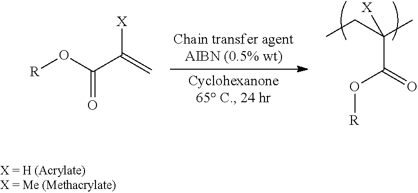

Moreover, their molecular weights can be controlled by use of conventional chain transfer agents (CTAs).

All monomers, except carvone acrylate (see below), provided linear polymers when polymerised in the presence of different concentrations of a chain transfer agent (dodecanemercaptan, DDM).

The right hand structure represents the inventors' current understanding of the structure obtained:

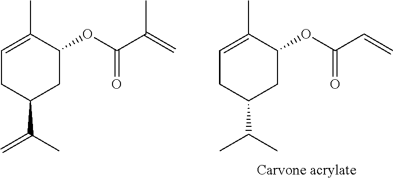

Molecular weights and glass transition temperatures obtained for each polymer when synthesised in presence of different concentrations of DDM are set out in Table 1.

In this regard, Table 1 shows the molecular weights and glass transition temperatures obtained for each polymer when using 0.5%, 1% and 5% of DDM.

The carvone acrylate monomer showed a very different reactivity leading to crosslinked materials and this is discussed in more detail later on in this document.

The key breakthrough is that the polymers produced give a range of glass transition temperatures (valuable for mechanical properties).

As expected, the acrylate monomers have considerably lower glass transition temperatures than their methacrylate analogues. This can be explained by the fact that the acrylate polymers have an α-hydrogen next to the carbonyl group, while the methacrylate analogues have a methyl group instead. (See below example of acrylate and methacylate polymer structures).

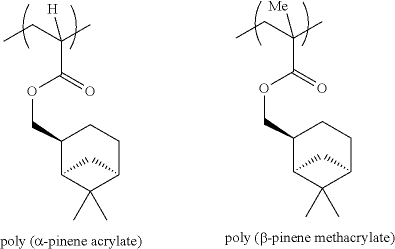

The below structures represent the inventors' current understanding of the structures obtained:

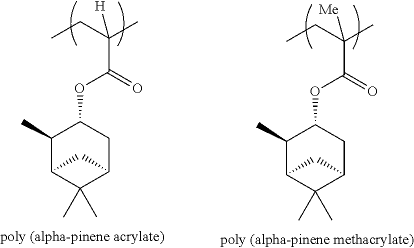

Therefore, acrylates have certain rotational freedom around the polymer chain, which is hindered in methacrylate polymers.

The T_{g} values are also affected by the size of the terpene moiety hanging in the polymer chain. As this group becomes larger, the polymer chains are pushed further apart, creating additional free volume.

The T_{g} values are seen to decrease in the order:

α-pinene>carvone>β-pinene>limonene (See Table 1).

TABLE 1

| | X = H (ACRYLATE) | | | | | | X = Me (METHACRYLATE) | | | | | |
|---|---|---|---|---|---|---|---|---|---|---|---|---|
| | 0.5% | | 1% | | 5% | | 0.5% | | 1% | | 5% | |
| % DDM | Mn | Tg | Mn | Tg | Mn | Tg | Mn | Tg | Mn | Tg | Mn | Tg |
| 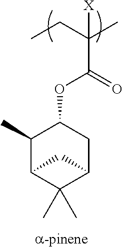 | 13,100^{a} | 71 | 13,400^{a} | 63 | 4,200^{b} | 12 | 16,000^{a} | 140 | 33,000^{b} 15,800^{a} | 142 | 3,800^{a} | 85 |
| 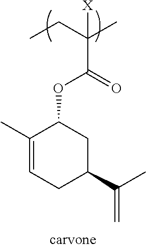 | Branched/Crosslinked polymers | | | | | | 12,500^{a} | 96^{c} | 30,000^{b} 14,800^{a} | 117 | 5,100^{a} | 90 |
| 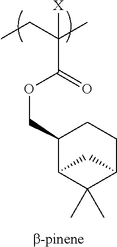 | 16,200^{a} | 33^{c} | 10,300^{a} | 41 | 4,800^{b} 1,700^{a} | −8 | 15,000^{a} | 115 | 26,000^{b} 13,600^{a} | 101 | 3,800^{a} | 65 |
| 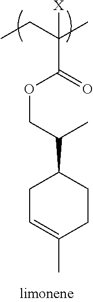 | 9,100^{a} | −9 | 7,400^{a} | −10 | 5,100^{b} | −18 | | | | | | |

NOTE:
^{a}Mn obtained in CHCl₃
^{b}Mn obtained in THF
^{c}These glass transition temperatures need to be reanalysed

Updated data for these experiments are provided in the Examples section.

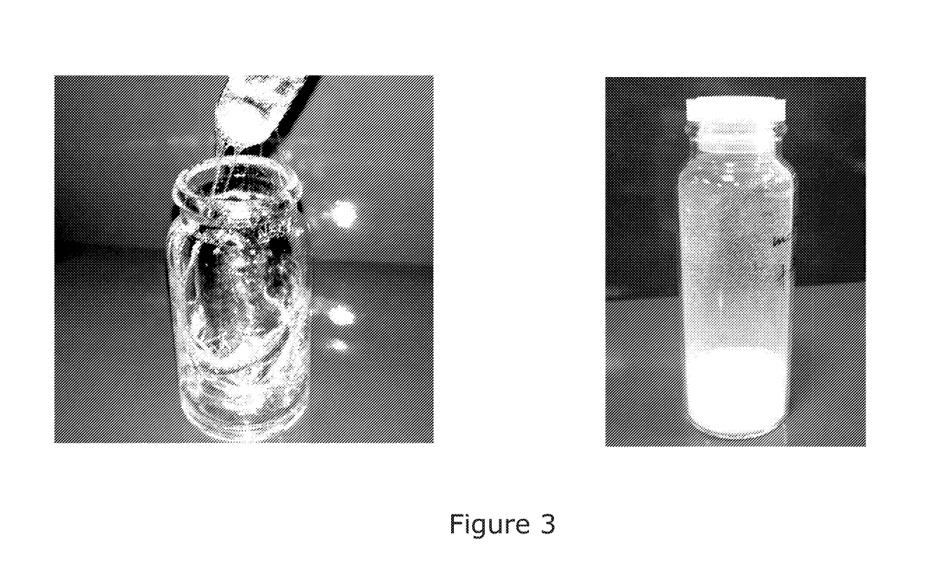

Examples of an acrylate with low T_{g} and a methacrylate with high T_{g} are shown in FIG. 3.

This is a picture of:
poly (β-pinene acrylate), Mn=4,800; Tg=−8° C. (left); and
poly (β-pinene methacrylate), Mn=4000, Tg=90° C. (right).

The acrylate polymer has a T_{g} below room temperature, −8° C.; therefore is viscous and sticky.

However the polymethacrylate shown in the right side of the picture has a T_{g} of around 90° C., so it is obtained as a fine white powder.

Further work has suggested the poly beta-pinene methacrylate may have a Tg closer to 85° C. than 90° C.; however it remains the case that the methacrylate derivative has a higher Tg than the acrylate derivative.

### Terpinolene and Gamma-Terpinene as Chain Transfer Agents

These two terpenes have shown a much smaller chain transfer constant with MMA than conventional chain transfer agents.

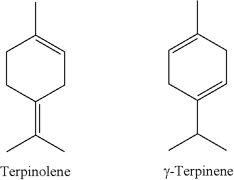

In theory, this would allow us to obtain polymers with higher molecular weights in a controlled way.

Table 2 shows molecular weights and conversions obtained when using terpinolene and gamma-terpinene as controlling agents.

In this regard, in Table 2, the molecular weights (Mn) and conversions (C %) obtained for the monomers tested are shown, next to the results obtained with dodecanemercaptan in order to compare. In every case, the concentration of chain transfer agent added was 1%.

As for the conversions, these are much lower when using terpenes as CTAs. When tested with other monomers (MMA, MA, Sty, VAc), terpinolene and terpinene retarded the polymerisation as well. The polymerisation conditions need to be optimised so that higher conversions are reached (higher T, different initiator) for the terpene-based monomers.

TABLE 2

| | X = H (ACRYLATE) | | | | | | | | X = Me (METHACRYLATE) | | | |
|---|---|---|---|---|---|---|---|---|---|---|---|---|
| | DDM | | Terpinolene | | γ-Terpinene | | DDM | | Terpinolene | | γ-Terpinene | |
| 1% CTA | Mn | C % | Mn | C % | Mn | C % | Mn | C % | Mn | C % | Mn | C % |
| 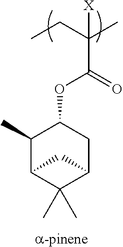 | 13,400 | 97 | 15,000 | 45 | 17,000 | 64 | 15,800 | 95 | In progress | | | |
| 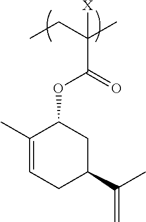 | Branched/Crosslinked polymers | | | | | | 14,800 | 99 | | 65 | | 78 |
| 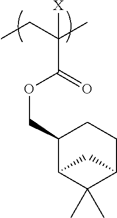 | 10,300 | 98 | 12,000 | 67 | 13,000 | 67 | 13,600 | 99 | | 78 | | 84 |
| 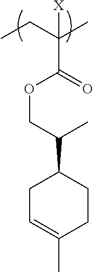 | 7.400 | 75 | | | | | | | | | | |

NOTE:
CTA: Chain Transfer Agent
DDM: Dodecanemercaptan
C %: Conversion (by 1H-NMR)

Updated data for these experiments are provided in the Examples section.

Some of these monomers are potential candidates for producing cross-linked coatings. The carvone acrylate and methacrylate derivatives have two polymerisable double bonds, as opposed to only one for the other terpene-based monomers:

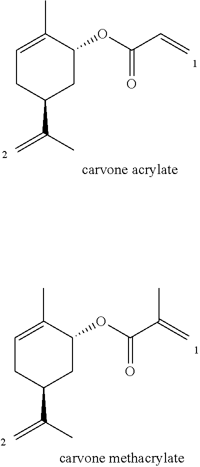 [SMILES: C=CC(=O)O[C@@H]1C[C@@H](C(=C)C)CC=C1C ; C=C(C)C(=O)O[C@@H]1C[C@@H](C(=C)C)CC=C1C]

The below structures represent the inventors' current understanding of the structures obtained:

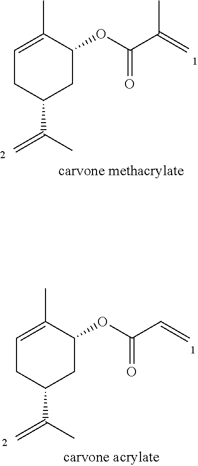 [SMILES: C=C(C)C(=O)O[C@@H]1C[C@H](C(=C)C)CC=C1C ; C=CC(=O)O[C@@H]1C[C@H](C(=C)C)CC=C1C]

These two monomers show different reactivity. Carvone methacrylate provides linear polymers (double bond 2 is not involved in the polymerisation). However, carvone acrylate provides branched/crosslinked polymers.

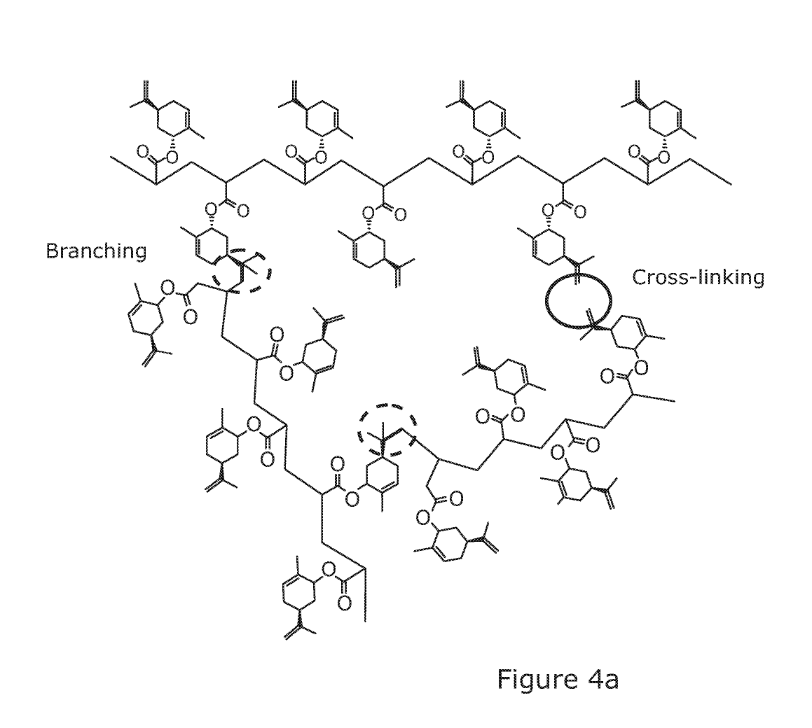

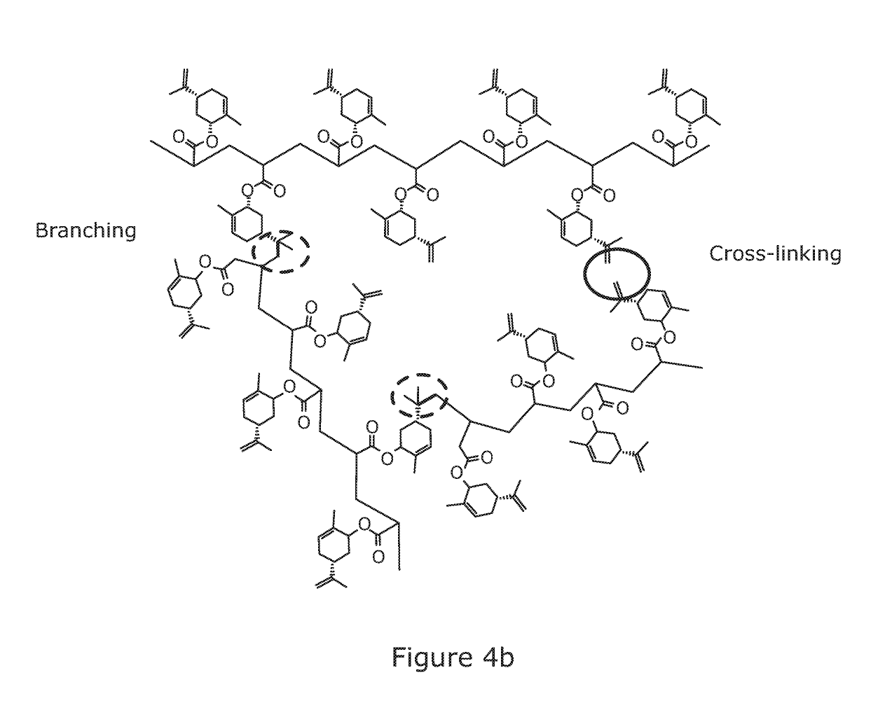

FIG. 4a shows the structure of the branched poly (carvone acrylate). See FIG. 4b for a version of this structure that reflects the inventors' current understanding of the structure obtained.

It can be seen that the chemical structure of the polymer obtained from carvone-based acrylate monomer is branched.

The double bond marked as 2 above is less reactive than the acrylate double bond, but it does react, creating branches as shown in FIG. 4. Due to the high density of double bonds, crosslinking occurs eventually, which means that the polymerisation needs to be stopped at certain conversion in order to avoid crosslinking.

The best conditions found so far are:

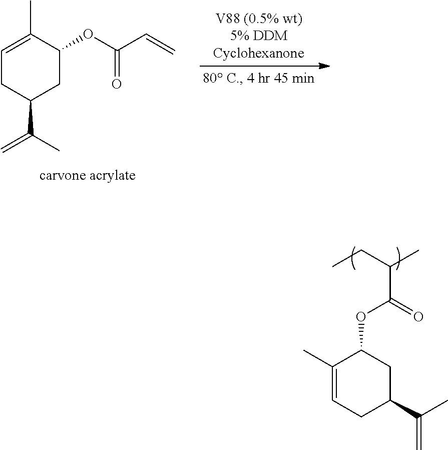

The below reaction scheme represents the inventors' current understanding of the structures used and obtained:

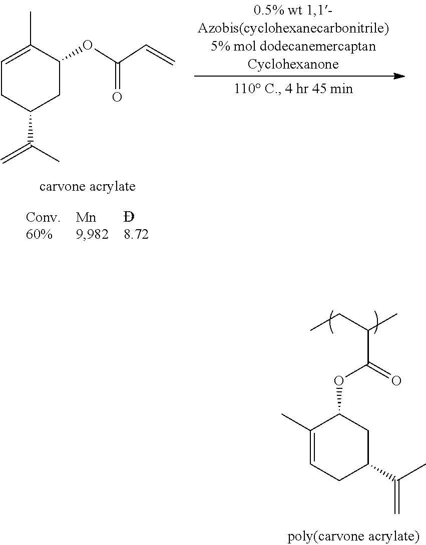

The present invention will be further described by reference to the following non-limiting examples.

### EXAMPLES

### Example 1

### Formation of Functionalised Terpenes and Terpenoids

### General Methods

### Experimental Procedures

Unless otherwise stated, reagents were purchased from commercial sources and used without further purification. All reactions were carried out in flame-dried glassware under Ar atmosphere. THF was distilled from Na/benzophenone immediately prior to use. DCM was dried over 4 Å molecular sieves prior to use. Methyl-tetrahydrofuran was purchased from Aldrich over 4 Å molecular sieves. Column chromatography was carried out either manually on silica gel Fluka 60 or on a Biotage® SP4 using Biotage® SNAP KP-Sil cartridges and petroleum ether (40-60° C.)/ethyl acetate as eluent, whilst monitoring by UV (254 nm) and thin layer chromatography (PMA stain). All NMR spectra were obtained in CDCl₃ at room temperature using Bruker® DPX300, Bruker® AV400 spectrometers for which chemical shifts are expressed in ppm relative to the solvent and coupling constants are expressed in Hz. Infrared spectroscopic data were recorded using a Bruker® Tensor27 FTIR spectrometer. Mass spectral data (and HRMS) were obtained using a Bruker® MicroTOF spectrometer. Optical rotations were measured on an ADP440 Polarimeter. Melting points were measured on a Gallenkamp® apparatus.

### General Method 1a: Forming Hydroxylated Derivatives

To a cold solution (0° C.) of terpene/terpenoid in solvent and a borating agent system was added dropwise and the mixture was stirred at 0° C. for 1 hour. After that time, keeping the solution at 0° C., solvent and an oxidising agent system were added subsequently in a drop wise manner. The resulting mixture was stirred for 1 hour warming to room temperature and additionally two hours at 80° C. The reaction was then allowed to cool before quenching by addition of a saturated aqueous solution of NH₄Cl. Aqueous layer was extracted with Et₂O (3×). Combined organic layers were washed with brine, dried and solvent was evaporated to yield the product.

### General Method 1b: Forming Hydroxylated Derivatives

To a cold solution (−78° C.) of terpene/terpenoid in solvent and a hydroalumination system was added dropwise and the mixture was stirred for 3 hours, warming to room temperature. The reaction was quenched by slow addition of a quenching system. The aqueous layer was extracted with Et₂O (3×). Combined organic layers were washed with brine, dried and solvent was evaporated to yield the product.

### General Method 1c: Forming Hydroxylated Derivatives

To a cold solution (0° C.) of 9-BBN in solvent was added dropwise a solution of terpene/terpenoid in solvent and the solution was stirred for 2 h warming to room temperature. Then the mixture was cooled down to 0° C. and base and an oxidising agent system were added simultaneously in a drop wise manner. After addition is completed the mixture was stirred for 2 h warming to 40° C. Then the solution was transferred to a separating funnel and the aqueous phase was extracted with Et₂O (3×). Combined organic layers were dried and solvent was evaporated. The resulting residue was filtered through a silica path washing with a 30% solution of petroleum ether/EtOAc. The solvent was removed to obtain the product.

### General Method 2: Esterification to Form Acrylate and Methacrylate Derivatives.

To a cold solution (0° C.) of the hydroxylated derivative in solvent was added base and acrylating agent and the mixture was stirred during 24 h warming to room temperature. The reaction was quenched by addition of a saturated aqueous solution of NaHCO₃. Aqueous phase was extracted with DCM (3×). Combined organic layers were washed with brine, dried and solvent was evaporated. The resulting residue was purified by chromatography on a Biotage SP4 eluting with 0-5% petroleum ether/ethyl acetate over 10 column volumes to isolate the product.

### Example 1a

### Production of Functionalised Monomers

### ((1R,2S,5R)-6,6-Dimethylbicyclo[3.1.1]heptan-2-yl)methanol

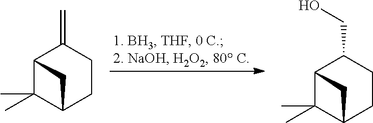

General method 1a was used.

Reagents and amounts used:
β-pinene (24 mL, 148 mmol)
Solvent and borating agent system: THF (56 mL), BH₃*SMe₂ (74 mL, 148 mmol, 2M in THF)
Solvent and oxidising agent system: EtOH (72 mL), NaOH (80 mL, 1M in H₂O),
H₂O₂ (36 mL, 30% v/v in H₂O)

Product obtained: 23.5 g (99% yield) of ((1R,2S,5R)-6,6-dimethylbicyclo[3.1.1]heptan-2-yl)methanol as a colorless oil that becomes solid after storing at low temperature.

Stereochemical assignment (based on literature precedent—G G. Giacomelli, L. Lardicci, F. Palla J. Org. Chem. 1984, 49, 310)

[α]_{D}²² −20 (c 4.9, CHCl₃). ¹H-NMR (400 MHz, CDCl₃): δ=3.6-3.5 (m, 2H), 2.4-2.3 (m, 2H), 2.3-2.2 (m, 1H), 2.0-1.9 (m, 1H), 1.9-1.8 (m, 4H), 1.5-1.4 (m, 1H), 1.15 (s, 3H), 0.94 (s, 3H), 0.93 (d, J=9.6 Hz, 1H). ¹³C-NMR (100 MHz, CDCl₃): δ=67.6 (t), 44.3 (d), 42.8 (d), 41.4 (d), 38.5 (s), 33.1 (t), 27.9 (q), 25.9 (t), 23.2 (q), 18.7 (t). HRMS (ESI-MS): calcd. for C₁₀H₁₈NaO: 177.1250. found: 177.1253.

### (1R,2R,3R,5S)-2,6,6-Trimethylbicyclo[3.1.1]heptan-3-ol

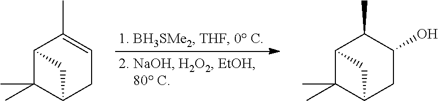

General method 1a was used.

Reagents and amounts used:
α-pinene (5.8 mL, 37.0 mmol)
Solvent and borating agent system: THF (14 mL), BH₃*SMe₂ (27.5 mL, 55.0 mmol, 2M in THF)
Solvent and oxidising agent system: EtOH (18 mL), NaOH (20 mL, 1M in H₂O), H₂O₂ (9 mL, 30% v/v in H₂O)

Product obtained: 4.95 g (87% yield) of (1R,2R,3R,5S)-2,6,6-trimethylbicyclo[3.1.1]heptan-3-ol as a colourless oil that becomes solid after storing at low temperature.

Stereochemical assignment (based on literature precedent—G. Zweifeil, H. C. Brown J. Am. Chem. Soc. 1964, 86, 393)

[α]_{D}²² −1.4 (c 3.4, CHCl₃). ¹H-NMR (400 MHz, CDCl₃): δ=4.1-4.0 (m, 1H), 2.49 (dd, J=15.8, 7.4 Hz, 1H), 2.35 (dt, J=9.7, 6.4 Hz, 1H), 2.0-1.9 (m, 4H), 1.78 (t, J=5.9 Hz, 1H), 1.69 (ddd, J=13.9, 4.7, 2.6 Hz, 1H), 1.20 (s, 3H), 1.11 (d, J=7.4 Hz, 3H), 0.90 (s, 3H). ¹³C-NMR (100 MHz, CDCl₃): δ=71.6 (d), 47.8 (d), 47.7 (d), 41.7 (d), 39.0 (t), 38.1 (s), 34.3 (t), 27.6 (q), 23.6 (q), 20.7 (q). HRMS (ESI-MS): calcd. for C₁₀H₁₈NaO: 177.1250. found: 177.1248.

### (1R,5S)-2-Methyl-5-(prop-1-en-2-yl)cyclohex-2-enol

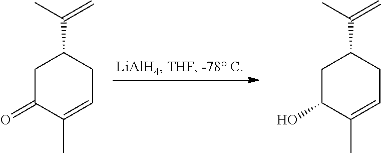

General method 1b was used.

Reagents and amounts used:
carvone (13.0 mL, 83.3 mmol)
solvent and a hydroalumination system: THF (250 mL) LiAlH₄ (100 mL, 100 mmol, 1M in THF)
quenching system: water (25 mL), aqueous solution of NaOH (50 mL, 1M in H₂O) and water (75 mL).

Product obtained: 12.6 g (99% yield) of (1R,5S)-2-methyl-5-(prop-1-en-2-yl)cyclohex-2-enol as a colourless oil that becomes solid after storing at low temperature.

Stereochemical assignment (based on literature precedent—L. Garver, P. Eikeren J. Org. Chem. 1976, 41, 2773)

[α]_{D}²² −40 (c 2.6, CHCl₃). ¹H-NMR (400 MHz, CDCl₃): δ=5.50 (ddt, J=5.2, 2.6, 1.4 Hz, 1H), 4.73 (m, 2H), 4.19 (bs, 1H), 2.3-2.2 (m, 1H), 2.2-2.1 (m, 1H), 2.1-2.0 (m, 1H), 2.0-1.9 (m, 1H), 1.76 (td, J=2.5, 1.4 Hz, 3H), 1.74 (s, 3H), 1.6-1.5 (m, 2H). ¹³C-NMR (100 MHz, CDCl₃): δ=148.9 (s), 136.2 (s), 123.7 (d), 109.0 (t), 70.8 (d), 40.4 (d), 37.9 (t), 31.0 (t), 20.5 (q), 18.9 (q). HRMS (ESI-MS): calcd. for C₁₀H₁₆NaO: 175.1093. found: 175.1091.

### ((1R,2S,5R)-6,6-Dimethylbicyclo[3.1.1]heptan-2-yl)methyl acrylate

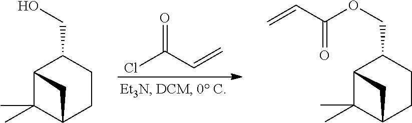

General method 2 was used.

Reagents and amounts used:
((1R,2S,5R)-6,6-dimethylbicyclo[3.1.1]heptan-2-yl)methanol (10.0 g, 64.8 mmol) in DCM (500 mL)
Base and acrylating agent: Et₃N (20 mL, 123 mmol) and acryloylchloride (9.5 mL, 97.2 mmol)

Product obtained: 9.00 g (66% yield) of a colourless oil identified as ((1R,2S,5R)-6,6-dimethylbicyclo[3.1.1]heptan-2-yl)methyl acrylate.

Stereochemical Assignment

[α]_{D}²² −10 (c 13.9, CHCl₃). ¹H-NMR (400 MHz, CDCl₃): δ=6.39 (dd, J=17.3, 1.5 Hz, 1H), 6.11 (dd, J=17.3, 10.4 Hz, 1H), 5.81 (dd, J=10.4, 1.5 Hz, 1H), 4.11 (dd, J=7.9, 2.8 Hz, 2H), 2.4-2.3 (m, 2H), 2.0-1.8 (m, 5H), 1.6-1.5 (m, 1H), 1.19 (s, 3H), 1.02 (s, 3H), 0.94 (d, J=9.6 Hz, 1H). ¹³C-NMR (100 MHz, CDCl₃): δ=166.2 (s), 130.2 (t), 128.6 (d), 68.8 (t), 42.9 (d), 41.2 (d), 40.2 (d), 38.4 (s), 32.9 (t), 27.8 (q), 25.7 (t), 23.1 (q), 18.5 (t). IR ν(cm⁻¹): 3011, 2923, 1717, 1636, 1619, 1470, 1409, 1385, 1367, 1396, 1192, 1059, 985. HRMS (ESI-MS): calcd. for C₁₃H₂₀NaO₂: 231.1356. found: 231.1345.

### (1R,2R,3R,5S)-2,6,6-Trimethylbicyclo[3.1.1]heptan-3-yl acrylate

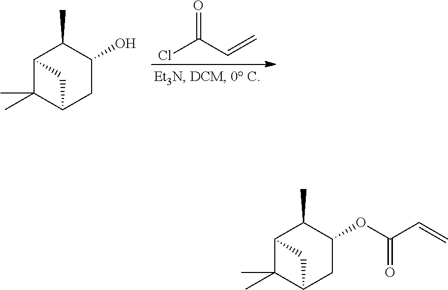

General method 2 was used.

Reagents and amounts used:
(1R,2R,3R,5S)-2,6,6-trimethylbicyclo[3.1.1]heptan-3-ol (4.90 g, 31.8 mmol) in DCM (310 mL)
Base and acrylating agent: Et₃N (8.86 mL, 63.5 mmol) and acryloylchloride (3.90 mL, 47.7 mmol)

Product obtained: 3.66 g (55% yield) of a colourless oil identified as (1R,2R,3R,5S)-2,6,6-trimethylbicyclo[3.1.1]heptan-3-yl acrylate.

Stereochemical Assignment

[α]_{D}²¹ −36 (c 2.2, CHCl₃). ¹H-NMR (400 MHz, CDCl₃): δ=6.38 (dt, J=17.3, 1.4 Hz, 1H), 6.12 (ddd, J=17.3, 10.4, 1.3 Hz, 1H), 5.79 (dt, J=10.4, 1.5 Hz, 1H), 5.11 (dt, J=9.4, 4.6 Hz, 1H), 2.6-2.5 (m, 1H), 2.4-2.3 (m, 1H), 2.2-2.1 (m, 1H), 2.0-1.9 (m, 1H), 1.82 (t, J=5.8 Hz, 1H), 1.69 (dt, J=14.4, 3.4 Hz, 1H), 1.22 (s, 3H), 1.10 (dd, J=7.4, 1.1 Hz, 3H), 1.07 (d, J=9.9 Hz, 1H), 0.96 (s, 3H). ¹³C-NMR (100 MHz, CDCl₃): δ=166.2 (s), 130.0 (t), 129.1 (d), 74.2 (d), 47.4 (d), 43.6 (d), 41.2 (d), 38.2 (s), 35.8 (t), 33.4 (t), 27.4 (q), 23.7 (q), 20.5 (q). IR ν(cm⁻¹): 3016, 2927, 1731, 1636, 1602, 1439, 1242. HRMS (ESI-MS): calcd. for C₁₃H₂₀NaO₂: 231.1356. found: 231.1350.

### (1R,5S)-2-Methyl-5-(prop-1-en-2-yl)cyclohex-2-enyl acrylate

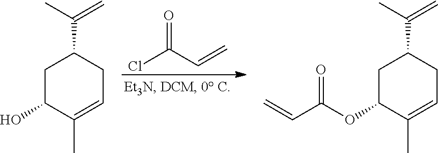

General method 2 was used.

Reagents and amounts used:
(1R,5S)-2-methyl-5-(prop-1-en-2-yl)cyclohex-2-enol (7.40 g, 48.6 mmol) in DCM (330 mL)
Base and acrylating agent: Et₃N (13.5 mL, 97.2 mmol) and acryloylchloride (5.94 mL, 72.9 mmol)

Product obtained: 9.1 g (91% yield) of a yellow oil identified as (1R,5S)-2-methyl-5-(prop-1-en-2-yl)cyclohex-2-enyl acrylate.

Stereochemical Assignment

[α]_{D}²² −44 (c 4.9, CHCl₃). ¹H-NMR (400 MHz, CDCl₃): δ=6.39 (d, J=17.3 Hz, 1H), 6.11 (dd, J=17.3, 10.4 Hz, 1H), 5.80 (d, J=10.4 Hz, 1H), 5.59 (d, J=4.2 Hz, 1H), 5.51 (bs, 1H), 4.70 (s, 2H), 2.4-2.3 (m, 1H), 2.3-2.2 (m, 1H), 2.1-2.0 (m, 1H), 2.0-1.9 (m, 1H), 1.69 (s, 3H), 1.62 (s, 3H). ¹³C-NMR (100 MHz, CDCl₃): δ=165.9 (s), 148.1 (s), 132.8 (s), 130.5 (t), 128.7 (d), 125.9 (d), 109.3 (t), 73.2 (d), 40.2 (d), 33.9 (t), 30.7 (t), 20.4 (q), 18.7 (q). IR ν(cm⁻¹): 3011, 2973, 2922, 1714, 1637, 1618, 1453, 1437, 1407, 1295, 1276, 1192, 1048, 985, 970. HRMS (ESI-MS): calcd. for C₁₃H₁₈NaO₂: 229.1199. found: 229.1196.

### (1R,5S)-2-Methyl-5-(prop-1-en-2-yl)cyclohex-2-enyl methacrylate

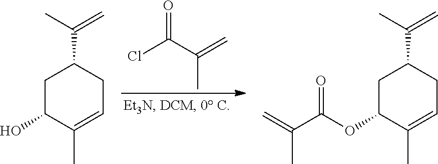

General method 2 was used.

Reagents and amounts used:
(1R,5S)-2-methyl-5-(prop-1-en-2-yl)cyclohex-2-enol (12.4 g, 83.0 mmol) in DCM (500 mL)
Base and acrylating agent: Et₃N (23.0 mL, 166 mmol) and methacryloylchloride (12.0 mL, 125 mmol)

Product obtained: 14.2 g (78% yield) of a yellow oil identified as (1R,5S)-2-methyl-5-(prop-1-en-2-yl)cyclohex-2-enyl methacrylate.

Stereochemical Assignment

[α]_{D}²² −44 (c 9.0, CHCl₃). ¹H-NMR (400 MHz, CDCl₃): δ=6.12 (s, 1H), 5.7-5.6 (m, 1H), 5.56 (s, 1H), 5.49 (bs, 1H), 4.73 (s, 2H), 2.4-2.3 (m, 1H), 2.24 (ddt, J 11.8, 6.0, 2.2 Hz, 1H), 2.2-2.1 (m, 1H), 2.1-2.0 (m, 1H), 1.96 (s, 3H), 1.72 (s, 3H), 1.65 (s, 3H), 1.6-1.5 (m, 1H). ¹³C-NMR (100 MHz, CDCl₃): δ=167.0 (s), 148.1 (s), 136.5 (s), 133.0 (s), 125.6 (d), 125.1 (t), 109.2 (t), 73.3 (d), 40.1 (d), 33.8 (t), 30.6 (t), 20.4 (q), 18.8 (q), 18.2 (q). IR ν(cm⁻¹): 3085, 3008, 1701, 1639, 1452, 1378, 1294, 1192, 1012. HRMS (ESI-MS): calcd. for C₁₄H₂₀NaO₂: 243.1356. found: 243.1354.

### ((1R,2S,5R)-6,6-Dimethylbicyclo[3.1.1]heptan-2-yl)methyl methacrylate

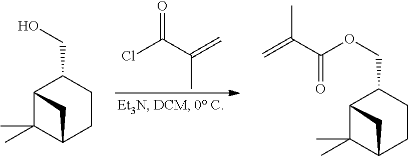

General method 2 was used.

Reagents and amounts used:
(1R,2S,5R)-6,6-dimethylbicyclo[3.1.1]heptan-2-yl)methanol (13.7 g, 89.0 mmol) in DCM (500 mL)
Base and acrylating agent: Et₃N (24 mL, 178 mmol) and methacryloylchloride (12.7 mL, 133 mmol)

Product obtained: 12.8 g (64% yield) of a light yellow oil identified as ((1R,2S,5R)-6,6-dimethylbicyclo[3.1.1]heptan-2-yl)methyl methacrylate.

Stereochemical Assignment

[α]_{D}²² −9.1 (c 3.8, CHCl₃). ¹H-NMR (400 MHz, CDCl₃): δ=6.09 (dd, J=1.7, 0.9 Hz, 1H), 5.54 (p, J=1.6 Hz, 1H), 4.09 (dd, J=8.0, 2.6 Hz, 2H), 2.5-2.4 (m, 2H), 2.0-1.8 (m, 5H), 1.94 (s, 3H), 1.19 (s, 3H), 1.02 (s, 3H), 1.0-0.9 (m, 2H). ¹³C-NMR (100 MHz, CDCl₃): δ=167.5 (s), 136.5 (s), 125.0 (t), 69.0 (t), 43.0 (d), 41.2 (d), 40.2 (d), 38.5 (s), 32.9 (t), 27.8 (q), 25.8 (t), 23.1 (q), 18.6 (t), 18.3 (q). IR ν(cm⁻¹): 2988, 2945, 2921, 1708, 1637, 1470, 1453, 1326, 1299, 1175. HRMS (ESI-MS): calcd. for C₁₄H₂₂NaO₂: 245.1512. found: 245.1510.

### (1R,2R,3R,5S)-2,6,6-Trimethylbicyclo[3.1.1]heptan-3-yl methacrylate

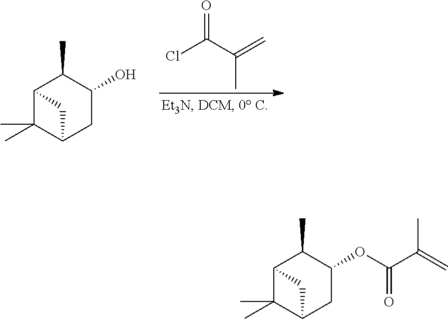

General method 2 was used.

Reagents and amounts used:
(1R,2R,3R,5S)-2,6,6-trimethylbicyclo[3.1.1]heptan-3-ol (11.4 g, 74.0 mmol) in DCM (500 mL)
Base and acrylating agent: Et₃N (20.6 mL, 148 mmol) and methacryloylchloride (10.7 mL, 111 mmol)

Product obtained: 7.76 g (47% yield) of a colourless oil identified as (1R,2R,3R,5S)-2,6,6-trimethylbicyclo[3.1.1]heptan-3-ylmethacrylate.

Stereochemical Assignment

[α]_{D}²² −24 (c 16.8, CHCl₃). ¹H-NMR (400 MHz, CDCl₃): δ=6.10 (dd, J=1.7, 0.9 Hz, 1H), 5.54 (p, J=1.6 Hz, 1H), 5.09 (ddd, J=9.2, 4.8, 4.2 Hz, 1H), 2.7-2.6 (m, 1H), 2.4-2.3 (m, 1H), 2.18 (qdd, J 7.4, 5.1, 2.3 Hz, 1H), 2.0-1.9 (m, 4H), 1.84 (td, J=5.9, 2.2 Hz, 1H), 1.71 (ddd, J=14.4, 3.9, 3.1 Hz, 1H), 1.23 (s, 3H), 1.12 (d, J=1.1 Hz, 3H), 1.09 (d, J=9.9 Hz, 1H), 0.98 (s, 3H). ¹³C-NMR (100 MHz, CDCl₃): δ=167.3 (s), 136.8 (s), 124.7 (t), 74.2 (d), 47.4 (d), 43.7 (d), 41.1 (d), 38.1 (s), 35.8 (t), 33.2 (t), 27.4 (q), 23.7 (q), 20.5 (q), 18.3 (q). IR ν(cm⁻¹): 2957, 2919, 1706, 1636, 1452, 1299, 1175, 1154. HRMS (ESI-MS): calcd. for C₁₄H₂₂NaO₂: 245.1512. found: 245.1513.

### (S)-2-((R)-4-Methylcyclohex-3-enyl)propyl acrylate

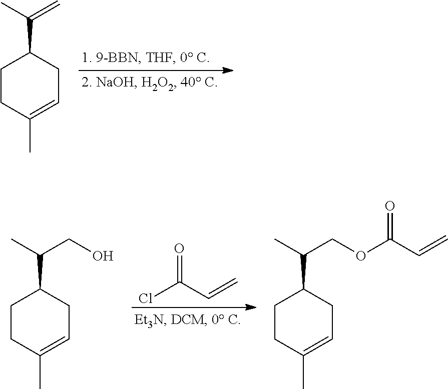

First Step:

General method 1c was used.

Reagents and amounts used:
limonene (11.9 mL, 73.4 mmol) in THF (29 mL)
9-BBN in solvent: 9-borabicyclo(3.3.1)nonane (191 mL, 95.4 mmol, 0.5 M in THF)
Base and oxidising agent system: NaOH (24 mL, 3 M in H₂O) and H₂O₂ (24 mL, 30% v/v in H₂O)

Second Step:

General method 2 was then used.

Reagents and amounts used:
yellow oil as obtained from first step, dissolved in DCM (500 mL)
Base and acrylating agent: Et₃N (13.5 mL, 97.2 mmol) and acryloylchloride (5.94 mL, 72.9 mmol)

Product obtained: 4.5 g (30% yield over two steps) of a colorless oil identified as (S)-2-((R)-4-Methylcyclohex-3-enyl)propyl acrylate (mixture of 2 diastereomers).

Stereochemical Assignment

¹H-NMR (400 MHz, CDCl₃): δ=6.38 (d, J=17.3 Hz, 1H), 6.10 (ddd, J=10.4, 8.5, 5.5 Hz, 1H), 5.80 (d, J=10.4 Hz, 1H), 5.35 (bs, 1H), 4.15 (dt, J=10.6, 5.1 Hz, 1H), 4.01 (dd, J=10.9, 1.0 Hz, 1H), 2.0-1.9 (m, 3H), 1.8-1.7 (m, 3H), 1.52 (s, 3H), 1.5-1.4 (m, 1H), 1.3-1.2 (m, 1H), 0.93 (dd, J=9.2, 6.9 Hz, 3H). ¹³C-NMR (100 MHz, CDCl₃): δ=166.3 (s), 134.0 (s), 133.9 (s), 130.4 (t), 128.6 (d), 120.5 (d), 120.4 (d), 67.8 (t), 67.7 (t), 36.8 (d), 36.7 (d), 35.6 (d), 35.5 (d), 30.6 (t), 30.5 (t), 29.5 (t), 27.8 (t), 26.9 (t), 25.5 (t), 23.4 (q), 14.0 (q), 13.62 (q). IR ν(cm⁻¹): 2989, 2925, 1713, 1470, 1408, 1386, 1028. HRMS (ESI-MS): calcd. for C₁₃H₂₀NaO₂: 231.1356. found: 231.1345.

### (S)-2-((R)-4-Methylcyclohex-3-enyl)propyl methacrylate

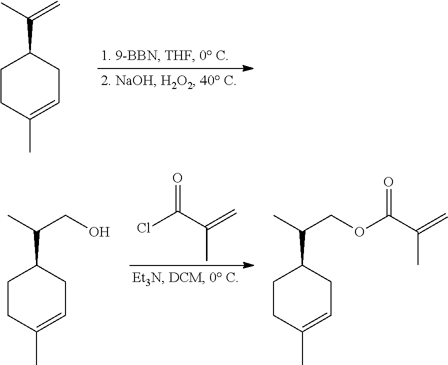

First Step:

General method 1c was used.

Reagents and amounts used:
limonene (11.9 mL, 73.4 mmol) in THF (29 mL)
9-BBN in solvent: 9-borabicyclo(3.3.1)nonane (191 mL, 95.4 mmol, 0.5 M in THF)
Base and oxidising agent system: NaOH (24 mL, 3 M in H₂O) and H₂O₂ (24 mL, 30% v/v in H₂O)

Second Step:

General method 2 was then used.

Reagents and amounts used:
yellow oil as obtained from first step, dissolved in DCM (500 mL)
Base and acrylating agent: Et₃N (20 mL, 146 mmol) and methacryloylchloride (10.6 mL, 109.5 mmol)

Product obtained: 13.4 g of a colorless oil identified as a mixture of (S)-2-((R)-4-methylcyclohex-3-enyl)propyl methacrylate and a methacryloyl chloride derivative.

This mixture can be separated by distillation so as to obtain the (S)-2-((R)-4-methylcyclohex-3-enyl)propyl methacrylate in pure form.

In this regard, limonene methacrylate is obtained together with an unsaturated impurity (a methacryloyl chloride derivative, as detected by NMR) that shows the same retention factor as the monomer in chromatographic columns. Therefore this impurity coelutes with the product.

The most common procedure to purify such monomers is by fractional distillation under reduced pressure. This can readily be achieved

In this regard, the major impurity is menth-1-en-9-al, which has a boiling point of 82-85° C. at 8 mmHg (Ref: Meinwald, J.; Jones, T. H., J. Am. Chem. Soc., 1978, 100, 1883-1886). Thus the impurity can be easily removed by reduced pressure distillation.

In order to avoid polymerisation reactions a radical inhibitor such as phenothiazine (PTZ) should be added to the mixture.

Results

a. Hydroboration/Oxidation of Terpenes

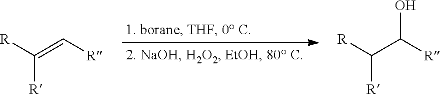

| Entry | Alkene | Borane | Alcohol | Yield (%)^{a} |
|---|---|---|---|---|
| 1 | 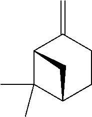 | BH₃*SMe₂ | 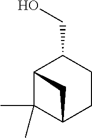 | 99 |
| 2 | 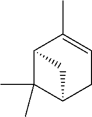 | BH₃*SMe₂ | 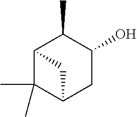 | 87 |
| 3 | 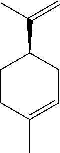 | 9-BBN | 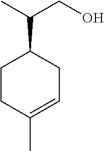 | —^{b} |

^{a}Isolated yields after purification. Multigram scale.
^{b}40° C. Complicated mixture. Alcohol used in the next step without further purification.

b. Hydroalumination of Carvone

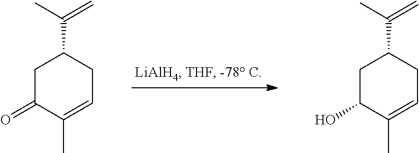

| Entry | Al-H | d.r. | Yield (%)^{a} |
|---|---|---|---|
| 1 | DIBAL-H | 5:1 | 99 |
| 2 | LiAlH₄ | 95:5 | 99 |

^{a}Isolated yields after purification. Multigram scale

To reduce the cost of the reduction of carvone it would be possible to lower the amount of LiAlH₄ from 1.0 equiv. to 0.3-0.5 equiv:

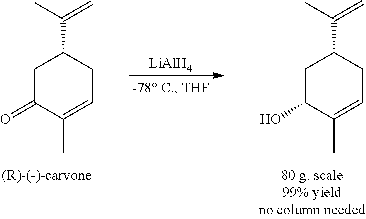

c. Acrylation/Methacrylation

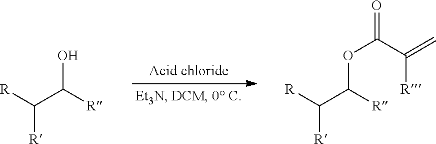

| Entry | Alcohol | Acid chloride | Acrylate | Yield (%)^{a} |
|---|---|---|---|---|
| 1 | 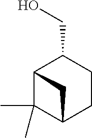 | 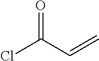 | 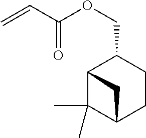 | 66 |
| 2 | 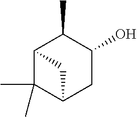 | 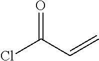 | 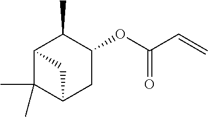 | 55 |
| 3 | 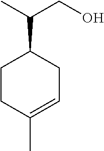 | 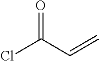 | 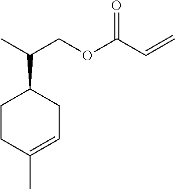 | 30^{b} |
| 4 | 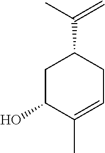 | 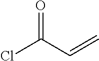 | 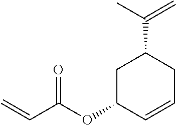 | 91 |
| 5 | 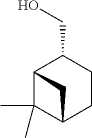 | 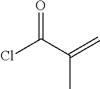 | 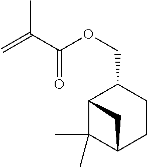 [SMILES: C=C(C)C(=O)OC[C@@H]1CC[C@H]2C[C@@H]1C2(C)C] | 64 |
| 6 | 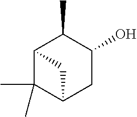 | 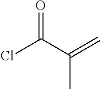 | 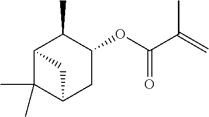 [SMILES: C=C(C)C(=O)O[C@@H]1C[C@@H]2C[C@H]([C@H]1C)C2(C)C] | 47 |
| 7 | 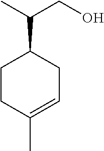 | 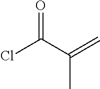 | 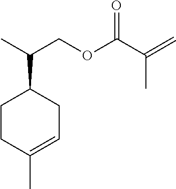 [SMILES: C=C(C)C(=O)OCC(C)[C@H]1CC=C(C)CC1] | —^{c} |
| 8 | 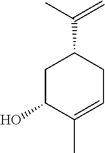 | 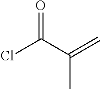 | 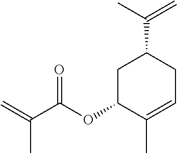 [SMILES: C=C(C)C(=O)O[C@@H]1C[C@H](C(=C)C)CC=C1C] | 78 |

^{a}Isolated yields after purification. Multigram scale.
^{b}Combined yield. 1:1 mixture of diastereomers (13C-NMR).
^{c}Impure compound - the required pure compound can be obtained by distillation.

### Example 1b

An alternative to the esterification process of general method 2 above was tested, to form acrylate and methacrylate derivatives. Specifically, the hydroxylated compounds were acrylated by treatment with the acrylic acid and T3P® as coupling agent, with Et₃N as a base.

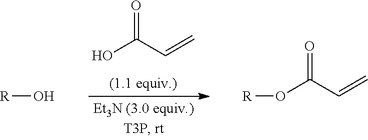

Table 3 shows the results for pinene alcohols with T3P®. Yields were improved as compared to general method 2 and the need for silica gel chromatography was avoided.

TABLE 3

| | | T3P | | | Yield/ |
|---|---|---|---|---|---|
| Starting material | Scale | (equiv.) | Solvent | Product | purification |
| 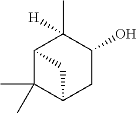 | 100 mg | 1.5 | CH₂Cl₂ | 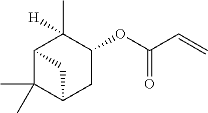 | 99% Aq. work-up |
| 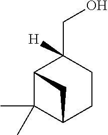 | 100 mg | 1.5 | CH₂Cl₂ | 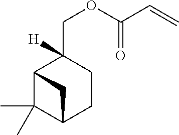 | 99% Aq. work-up |

The synthesis of the monomers was also carried out using this alternative process but using the environmentally friendly MeTHF as solvent, instead of CH₂Cl₂. The yields are set out in Table 4.

TABLE 4

| Starting material | Acryloyl chloride | Methacryloyl chloride |
|---|---|---|
| 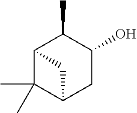 | 61% 52% (45g scale) | 33% |
| 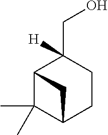 | 62% | 52% (30 g scale) |
| 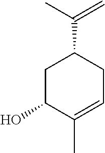 | 91% (50 g scale) | 92% (50 g scale) |

### Example 1c

### Further Examples

### Step 1: Hydroxylation

### Starting Materials Containing Carbonyl Groups

### Camphene

General method 1a was successfully used to hydroxylate camphene through a hydroboration/oxidation tandem protocol, as with the pinene materials above.

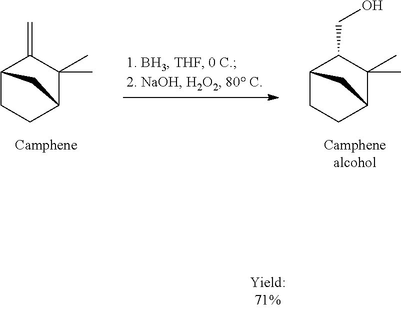

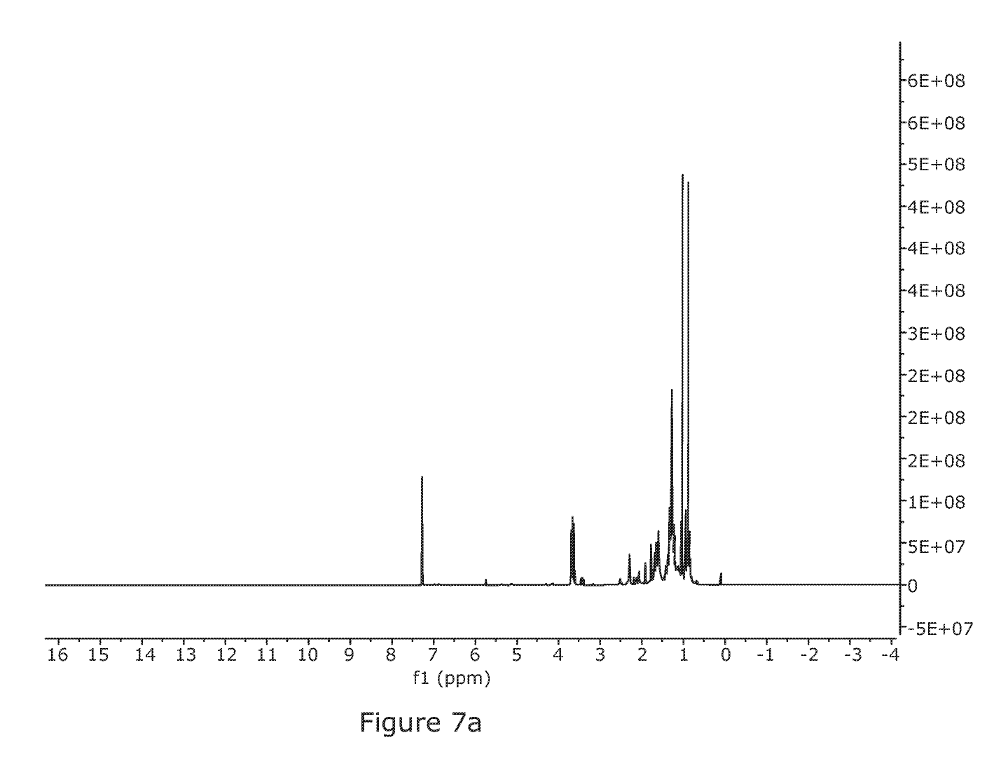

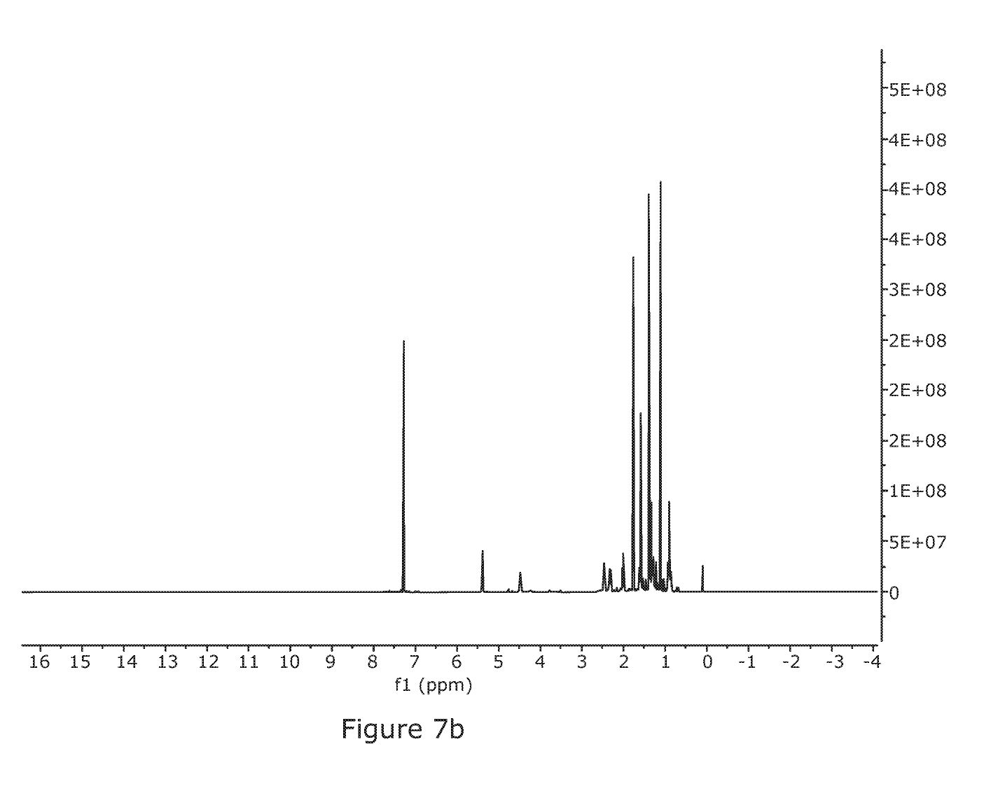

The 1H-NMR spectra for the camphene alcohol product as obtained is shown in FIG. 7a.

### Verbenone

General method 1b was successfully used to reduce verbenone to verbenone alcohol, as with carvone above.

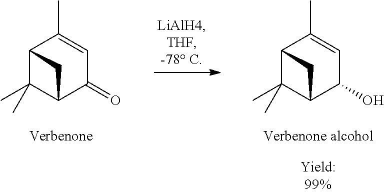

Reagents and amounts used:
verbenone (13.0 mL, 83.3 mmol)
solvent and a hydroalumination system: THF (250 mL) LiAlH₄ (100 mL, 100 mmol, 1M in THF)
quenching system: water (25 mL), aqueous solution of NaOH (50 mL, 1M in H₂O) and water (75 mL).

The 1H-NMR spectra for the verbenone alcohol product as obtained is shown in FIG. 7b.

It has therefore been proven that these two procedures work well, yielding alcohols in very good to excellent yields. Any terpene with a double bond could be hydroxylated via hydroboration using BH₃. If the terpene has more than one double bond, a more bulky boron agent (e.g. catechol borane or 9-BBN) can be used to selectively hydroxylate the less sterically hindered double bond, as was achieved for limonene.

In addition, terpenoids containing carbonyl groups (including verbenone or carvone) have been successfully reduced to alcohols using LiAlH₄, normally in quantitative yields.

### Starting Materials Containing Alcohol Groups

Terpenoids like menthol and terpineol already contain hydroxyl groups and therefore can be used directly in general method 2 in order to react with acrylic/methacrylic acid to form acrylate and methacrylate monomers.

### Step 2: Acrylation/Methacrylation

Acrylation reactions were successfully carried out using general method 2, and the results are shown below.

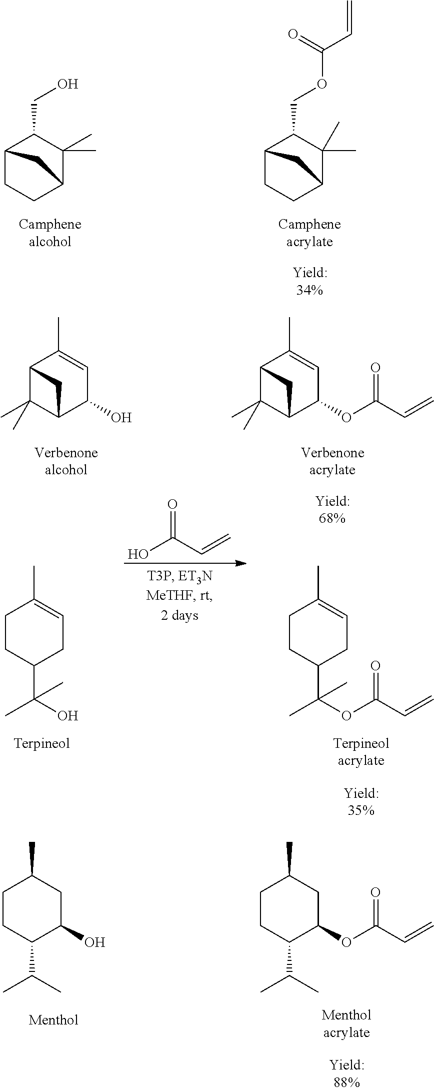

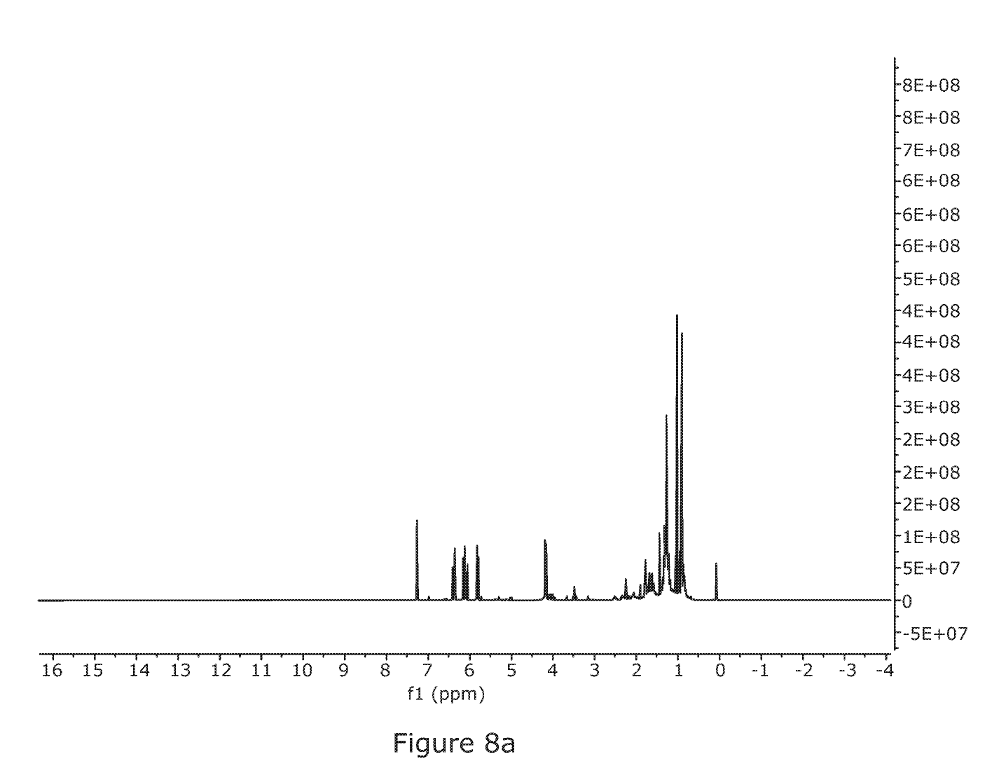

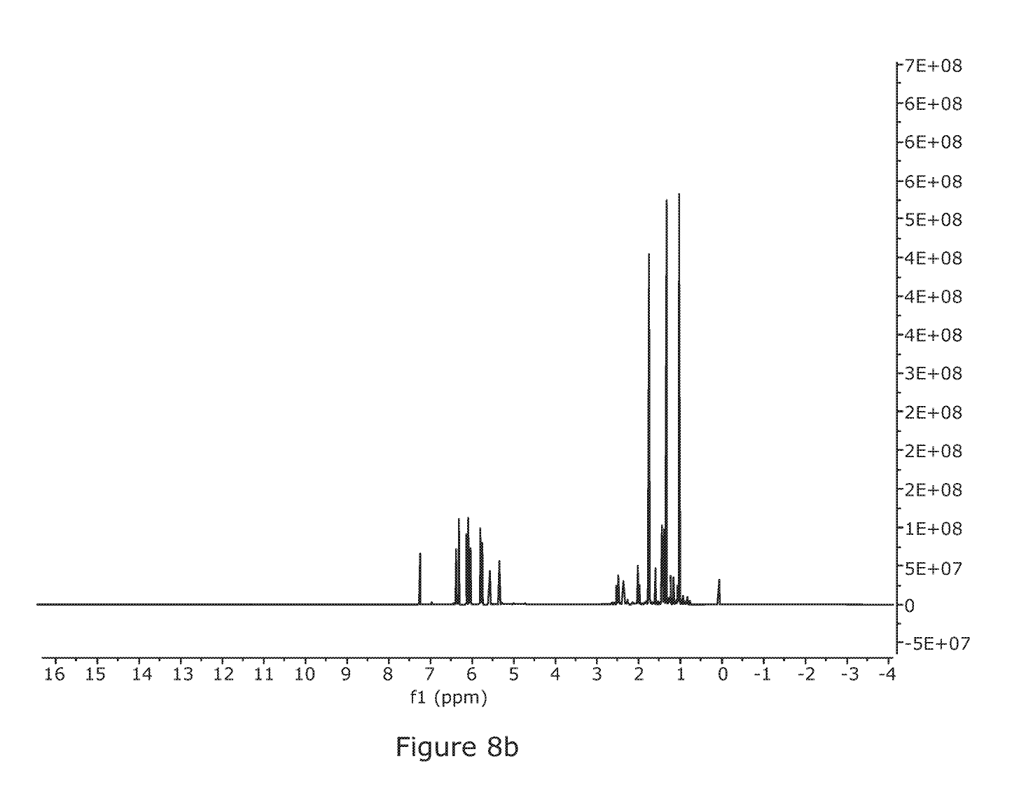

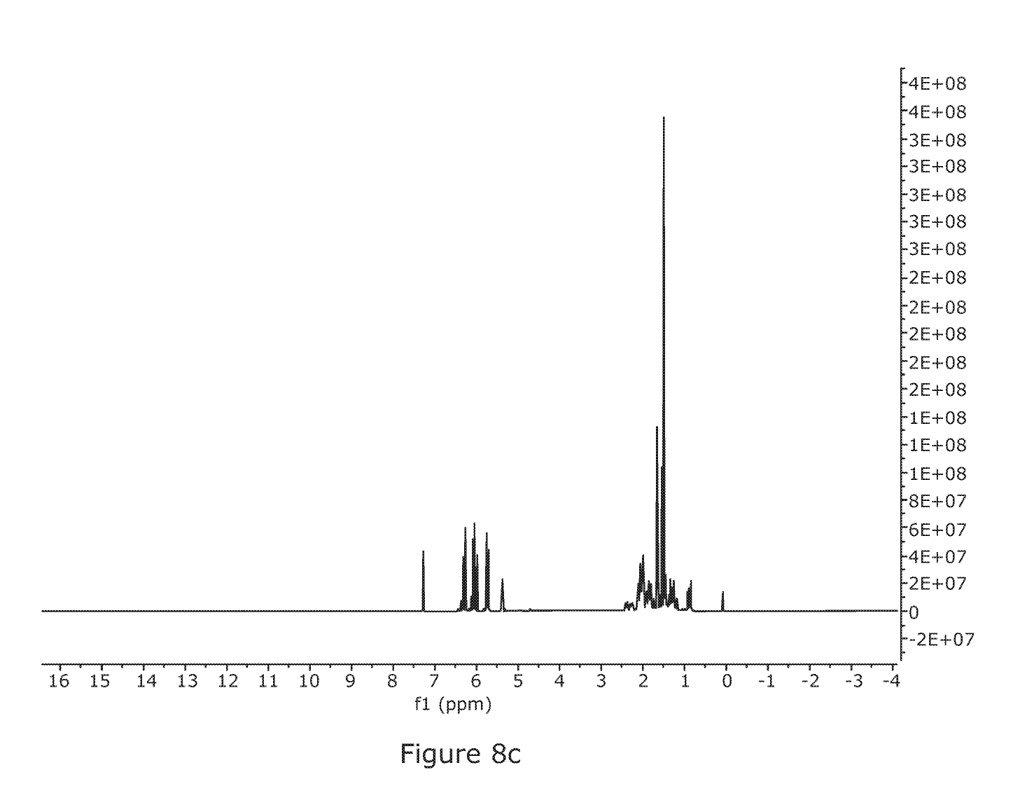

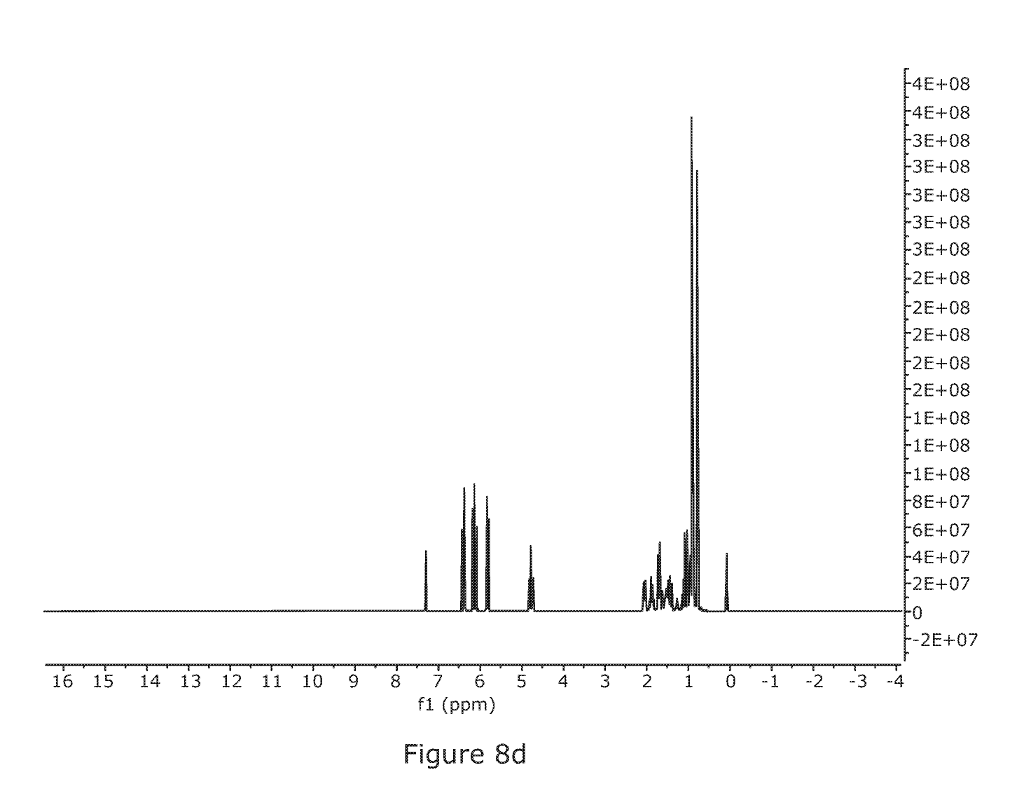

The 1H-NMR spectra for the acrylate products as obtained are shown in FIG. 8 (FIG. 8a=camphene acrylate; FIG. 8b=verbenone acrylate; FIG. 8c=terpineol acrylate; FIG. 8d=menthol acrylate).

The same conditions can be used for methacrylation.

### Scale Up

The quantities of product obtained by the two step processes of the invention (as per Examples 1a and 1b) are summarised in Table 5. This shows that quantities sufficient to proceed to polymerisation can be obtained.

TABLE 5

| SM | Product | Quantity |
|---|---|---|
| 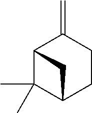 | 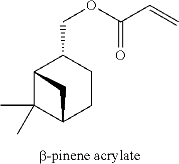 | 18.0 g |
| | 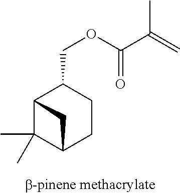 [SMILES: C=C(C)C(=O)OC[C@@H]1CC[C@H]2C[C@@H]1C2(C)C] | 21.5 g |
| 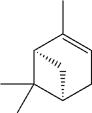 | 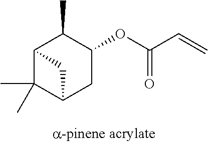 | 32.4 g |
| | 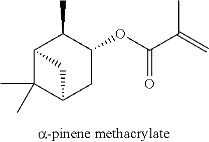 [SMILES: C=C(C)C(=O)O[C@@H]1C[C@@H]2C[C@H]([C@H]1C)C2(C)C] | 15.8 g |
| 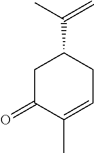 | 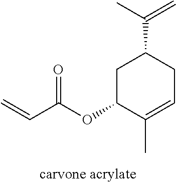 | 45 g |
| | 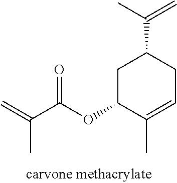 [SMILES: C=C(C)C(=O)O[C@@H]1C[C@H](C(=C)C)CC=C1C] | 49 g |

### Example 1d

Monomers which were acrylate and methacrylate derivatives of limonene were formed by a one-step process.

This was a catalytic approach based on the following schemes:

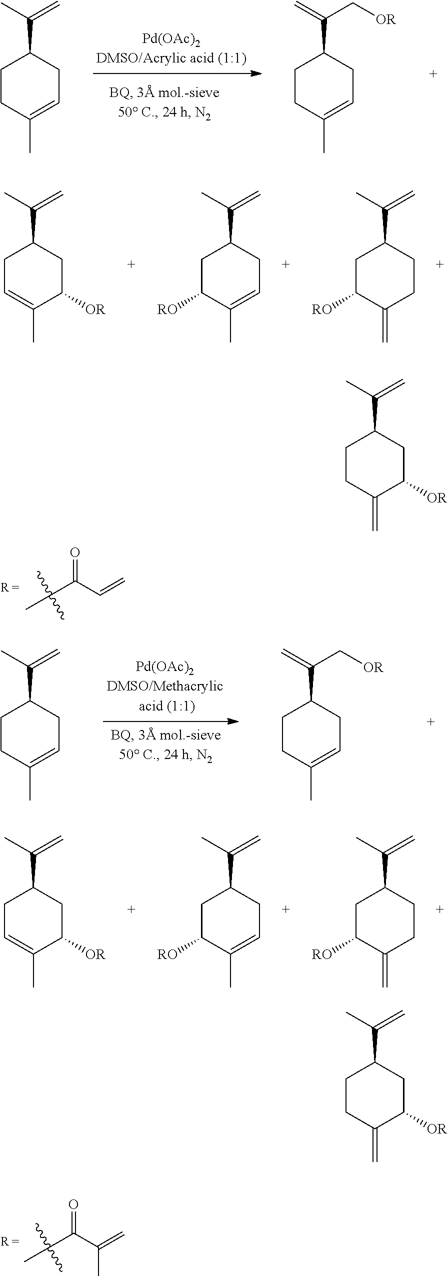

It will be appreciated that other R groups could be used.

BQ=1,4 benzoquinone

The reaction was carried out under nitrogen atmosphere. Conditions were as described in M. Czapiewski, M. A. R. Meier, Catal. Sci. Technol., 2014, 4, 2318.

The results for acrylic acid were:

TABLE 6

| Reagents | Solvent | Temp. | GC analysis |
|---|---|---|---|
| BQ (1.0 equiv.) | DMSO/Acrylic acid (1:1) | 90° C. | 3 products (4:3:1), |
| | | | 25% conversion |

The results for methacrylic acid (MA) were:

TABLE 7

| Reagents | Solvent | Temp. | GC analysis |
|---|---|---|---|
| BQ (1.0 equiv) | DMSO/MA (1:1) | 100° C., | 3 products (5:3:1), |
| | | MW | 30% conversion |

The reaction was also shown to work when Me-THF was used as solvent:

### Example 1e

### (3R)-6,6-dimethyl-2-methylenebicyclo[3.1.1]heptan-3-yl methacrylate and (6,6-dimethylbicyclo[3.1.1]hept-2-en-2-yl)methyl methacrylate

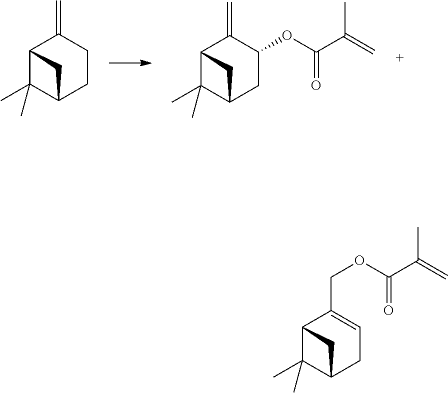

Monomers which were methacrylate derivatives of beta-pinene were formed by a one-step process.

β-pinene (2.00 g, 14.8 mmol) was dissolved in methacrylic acid (10 mL). Benzoquinone (3.20 g, 29.6 mmol) was added followed by Pd(OAc)₂ (80.0 mg, 2 mol %). The reaction was stirred under O₂ atmosphere for 72 hours at 50° C. The reaction mixture was allowed to cool down before flushing through Celite. The residue was diluted with toluene (10 mL) and the solvents were removed in vacuo. The crude product was purified on silica gel chromatography using petroleum ether as solvent system to give 10:1 mixture of (3R)-6,6-dimethyl-2-methylenebicyclo[3.1.1]heptan-3-yl methacrylate and (6,6-dimethylbicyclo[3.1.1]hept-2-en-2-yl)methyl methacrylate as yellow oil (2.67 g, 82% yield).

The ratio of the products did not change and the product was shown to be stable after standing at room temperature for 7 days in deuterated chloroform.

### Major product—(3R)-6,6-dimethyl-2-methylenebicyclo[3.1.1]heptan-3-yl methacrylate

δ_{H} (400 MHz, CDCl₃): 6.11 (1H, dd, J 0.88, 1.72 Hz), 5.64 (1H, d, J 8.04 Hz), 5.56 (1H, dt, J 1.61, 4.82 Hz), 5.10 (1H, t, J 1.17 Hz), 4.90 (1H, br. s), 2.56 (1H, t, J 5.55 Hz), 2.02 (1H, dddd, J 1.75, 4.09, 5.99, 11.55 Hz), 1.97 (3H, dd, J 1.0, 1.40 Hz), 1.85 (1H, ddd, J 0.88, 4.24, 15.05 Hz), 1.66 (1H, d, J 9.94 Hz), 1.31 (3H, br. s), 0.92-0.84 (2H, m), 0.73 (3H, s). δ_{C} (100 MHz, CDCl₃): 166.6 (q), 149.8 (q), 136.5 (q), 124.7 (CH2), 113.9 (CH2), 68.4 (CH), 50.4 (CH), 40.2 (q), 39.2 (CH), 32.9 (CH2), 27.4 (CH2), 25.5 (CH3), 21.6 (CH3), 18.0 (CH3).

### Minor product—(6,6-dimethylbicyclo[3.1.1]hept-2-en-2-yl)methyl methacrylate

For the minor product, the following peaks are discernible. The others overlap with the major product. δ_{H} (400 MHz, CDCl₃): 6.13-6.11 (1H, m), 5.62-5.58 (1H, m), 4.54 (2H, J 1.46 Hz), 2.34-2.28 (2H, m), 2.18-2.12 (2H, m), 1.60-1.40 (2H, m). δ_{C} (100 MHz, CDCl₃): 166.9 (q), 142.6 (q), 136.1 (q), 124.9 (CH2), 121.0 (CH), 66.8 (CH2), 43.2 (CH), 40.3 (CH), 37.7 (q), 31.1 (CH2), 30.9 (CH2), 25.8 (CH3), 22.8 (CH3), 20.7 (CH3). HRMS. Found: 243.1363; C₁₄H₂₀O₂ (M+Na⁺), requires 243.1356.

### Example 1f

### (3R)-6,6-dimethyl-2-methylenebicyclo[3.1.1]heptan-3-yl acrylate and (6,6-dimethylbicyclo[3.1.1]hept-2-en-2-yl)methyl acrylate

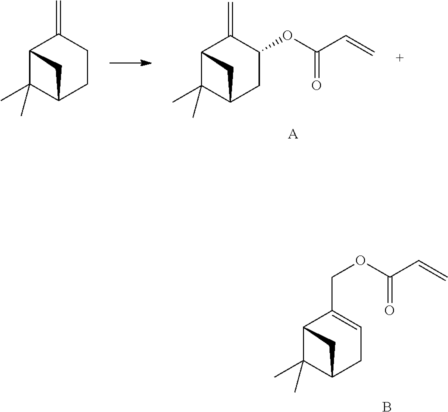

Monomers which were acrylate derivatives of beta-pinene were formed by a one-step process.

β-Pinene (2.00 g, 14.8 mmol) was dissolved in acrylic acid (8 mL). Benzoquinone (3.20 g, 29.6 mmol) was added followed by Pd(OAc)₂ (80.0 mg, 2 mol %). The reaction was stirred under O₂ atmosphere for 72 hours at 50° C. The reaction mixture was allowed to cool down before filtering through Celite and the solvent removed in vacuo. The excess of acrylic acid was removed by flushing the residue through pad of silica with petroleum ether. The solvent was removed in vacuo and the residue purified on silica gel chromatography (petroleum ether) to yield a 1:1 mixture of (3R)-6,6-dimethyl-2-methylenebicyclo[3.1.1]heptan-3-yl acrylate and (6,6-dimethylbicyclo[3.1.1]hept-2-en-2-yl)methyl acrylate as clear oil (1.36 g, 45% yield).

### Compound A—(3R)-6,6-dimethyl-2-methylenebicyclo[3.1.1]heptan-3-yl acrylate

δ_{H} (300 MHz, CDCl₃): 6.40 (1H, dd, J 1.60, 17.33 Hz), 6.13 (1H, ddd, J 2.45, 10.36, 17.33 Hz), 5.81 (1H, ddd, J 1.60, 2.92, 10.36 Hz), 5.65 (1H, d, J 7.91 Hz), 5.08 (1H, t, J 1.13 Hz), 4.90 (1H, br. s), 2.55 (1H, t, J 5.37 Hz), 2.48-2.35 (2H, m), 2.04-1.96 (1H, m), 1.83 (1H, ddd, J 0.94, 4.24, 14.98 Hz), 1.63 (1H, d, J 9.98 Hz), 1.29 (3H, s), 0.71 (3H, s). δ_{C} (100 MHz, CDCl₃): 166.3 (q), 150.4 (q), 130.8 (CH2), 128.8 (CH), 114.6 (CH2), 69.0 (CH), 51.0 (CH), 40.8 (q), 39.8 (CH), 33.5 (CH2), 28.1 (CH2), 26.1 (CH3), 22.2 (CH3).

### Compound B—(6,6-dimethylbicyclo[3.1.1]hept-2-en-2-yl)methyl acrylate

For the compound B, the following peaks are discernible. The others overlap with the compound A.

δ_{H} (300 MHz, CDCl₃): 6.40 (1H, dd, J 1.60, 17.33 Hz), 6.13 (1H, ddd, J 2.45, 10.36, 17.33 Hz), 5.81 (1H, ddd, J 1.60, 2.92, 10.36 Hz), 5.59 (1H, ddd, J 1.41, 2.92, 4.33 Hz), 4.54 (2H, ddd, J 1.41, 2.92, 3.01 Hz), 2.48-2.35 (1H, m), 2.34-2.24 (2H, m), 2.17-2.07 (2H, m), 1.29 (3H, s), 1.20 (1H, d, J 8.67 Hz), 0.83 (3H, s). δ_{C} (100 MHz, CDCl₃): 166.0 (q), 143.1 (q), 130.6 (CH2), 129.5 (CH), 121.9 (CH), 67.3 (CH2), 43.8 (CH), 40.9 (CH), 38.3 (q), 31.7 (CH2), 31.5 (CH2), 26.4 (CH3), 21.3 (CH3). HRMS. Found: 229.1187; C₁₃H₁₈O₂ (M+Na⁺), requires 229.1199.

### Example 1g

Monomers which were acrylate or methacrylate derivatives of alpha pinene were successfully formed by a one-step process in an analogous manner to Examples 1e and 1f.

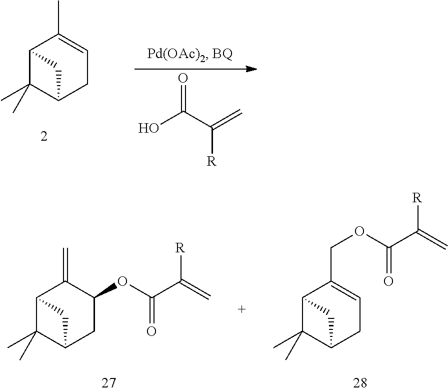

The reactions were:

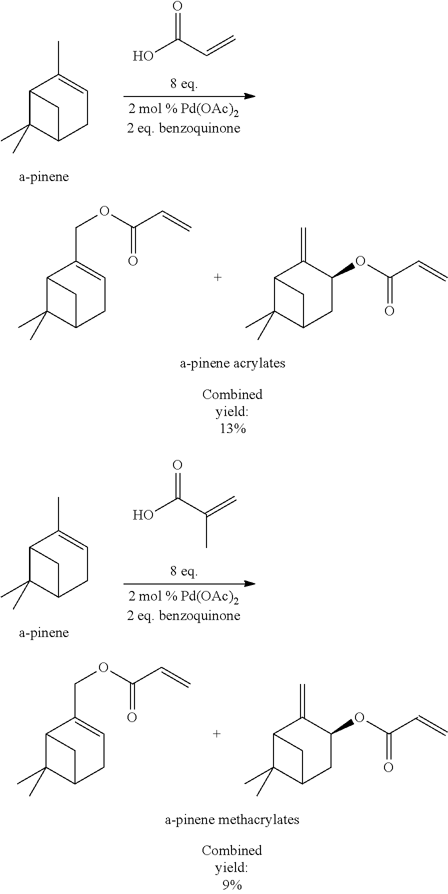

The reactions were conducted at 50° C. for 72 hr. The excess of (meth)acrylic acid was removed by forming an azeotrope with water or toluene and evaporating the azeotrope under reduced pressure. The resulting mix was purified in a silica chromatographic column using hexane and ethyl acetate as eluent.

### Example 1h

Monomers which were acrylate or methacrylate derivatives of limonene were successfully formed by a one-step process in an analogous manner to Examples 1e and 1f.

The reaction was carried out with (1R)-(+)-limonene 3 giving all 5 possible products:

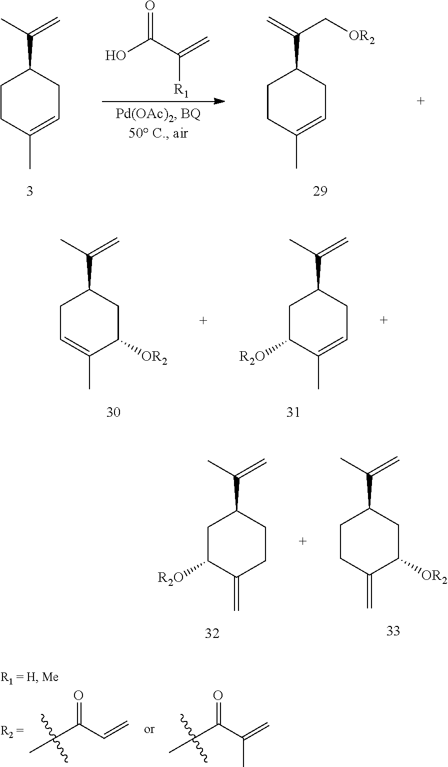

The Pd(OAc)₂/BQ complex that had been working and developed for β-pinene was used. The yield was 89%.

There was a complex mixture obtained which was non-separable by silica gel chromatography. However, the all the limonene acrylate or methacrylate isomers obtained in this mixture can be polymerised together. Therefore no separation prior to polymerisation was required.

The conditions of M. Czapiewski, M. A. R. Meier, Catal. Sci. Technol., 2014, 4, 2318 in DMSO were used with 0.5 mM concentration. This led to isolation of the major product 29 in a 5:1 ratio.

TABLE 8

| Catalyst/ligand | Atm | Solvent | Time | Temp. | Yield | Ratio |
|---|---|---|---|---|---|---|
| Pd(OAc)₂ | O₂ | AA (18.0 | 24 h | 50° C. | 89% | Complex |
| (2 mol %), | | equiv.) | | | | mixture |
| BQ (2.0 equiv.) | | | | | | |
| Pd(OAc)₂ | air | AA/DMSO | 24 h | 50° C. | 12% | 5:1 |
| (4 mol %), | | (1:1) | | | | (29/30) |
| BQ (2.0 equiv.) | | | | | | |

### Example 1i

### Further Examples

The method of Examples 1d and 1e was successfully used to form acrylate and methacrylate monomers from camphene, according to the reaction schemes below.

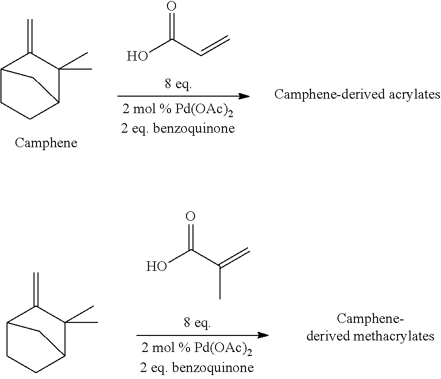

The reactions were conducted at 50° C. for 72 hr. The excess of (meth)acrylic acid was removed by forming an azeotrope with water or toluene and evaporating the azeotrope under reduced pressure. The resulting mix was purified in a silica chromatographic column using hexane and ethyl acetate as eluent.

### Scale Up

The quantities of each product obtained by the one-step catalytic route of the invention (as per Examples 1d-1h), are summarised in Table 9. This shows that quantities sufficient to proceed to polymerisation can be obtained.

TABLE 9

| SM | Product | Quantity |
|---|---|---|
| 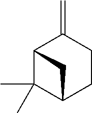 | 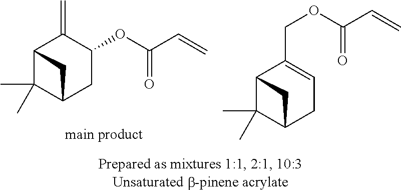 | 1.36 g (1:1 mixture) 3.4 g (2:1 mixture) 0.85 g (10:3 mixture) |
| | 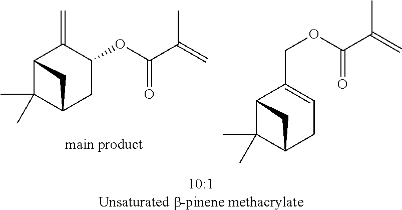 [SMILES: C=C(C)C(=O)O[C@@H]1C[C@H]2C[C@@H](C1=C)C2(C)C ; C=C(C)C(=O)OCC1=CC[C@H]2C[C@@H]1C2(C)C] | 4.4 g |
| 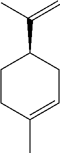 | 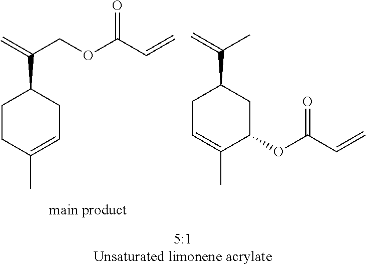 | 0.44 g |
| | 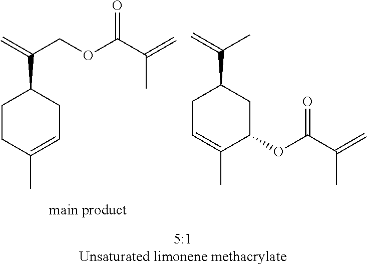 | 1 g |

### Example 2

### Formation of Polymers from the Functionalised Terpene/Terpenoid Monomers

In order to test the polymerisability of these new monomers, conventional free radical polymerisation (FRP) reactions were carried out.

### Example 2a

### Use of AIBN as Initiator

The polymerisation was carried out in the presence of 0.5% wt of azobisisobutyronitrile (AIBN) as initiator.

All reactions were carried out by mixing of 1 g of monomer, 0.5% wt of AIBN and 1.5 mL of toluene in a 10 mL round-bottomed flask. The mixture was previously degassed by freeze-pump-thaw technique and then heated up to 65° C. for 24 h.

The Conversion % was determined by ¹H NMR. The Mₙ (g mol⁻¹) and the M_{w}/Mₙ were determined by GPC-SEC in THF using PMMA standards.

Table 10 shows results from the FRP of the terpene-based acrylate and methacrylate derivatives.

TABLE 10

| | Conversion | | |
|---|---|---|---|
| Monomer | (%) | Mₙ (g mo1⁻¹) | M_{w}/Mₙ |
| 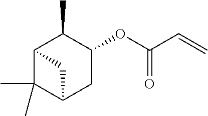 | ≧99 | Insoluble | Insoluble |
| 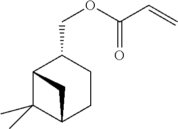 | 98 | 62,900 | 5.01 |
| 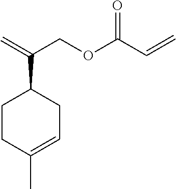 | 90 | 19,000 | 4.22 |
| 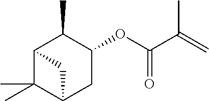 [SMILES: C=C(C)C(=O)O[C@@H]1C[C@@H]2C[C@H]([C@H]1C)C2(C)C] | ≧99 | Insoluble | Insoluble |
| 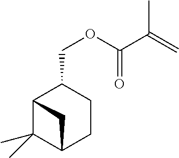 [SMILES: C=C(C)C(=O)OC[C@@H]1CC[C@H]2C[C@@H]1C2(C)C] | 98 | 106,500 | 5.82 |
| 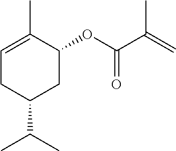 [SMILES: C=C(C)C(=O)O[C@@H]1C[C@H](C(C)C)CC=C1C] | ≧99 | 52,300 | 4.38 |

### Example 2b

### Use of V88 as Initiator

A range of conditions was tested to study the polymerisability of the carvone acrylate monmer, using 1,1′-azobis(cyclohexane-1-carbonitrile) (V88) as initiator. The half-life time of V88 is just under 10 h at 90° C., and 1 h at 110° C.

All reactions were carried out by mixing of 1 g of monomer, 0.5% wt of initiator and 5% of dodecanemercaptan chain transfer agent (DDM) in 1.5 mL of cyclohexanone in a 10 mL round-bottomed flask at 110° C. The mixture was previously degassed by freeze-pump-thaw technique and then heated up to the given temperatures.

The Conversion % was determined by ¹H NMR. The Mₙ (g mol⁻¹) and the M_{w}/Mₙ were determined by GPC-SEC in THF using PMMA standards.

Table 11 shows results from the FRP of the carvone-based acrylate derivative.

TABLE 11

| | T | | | Conversion | | |
|---|---|---|---|---|---|---|
| Initiator | (° C.) | t (h) | [DDM] | (%) | Mₙ (g mol⁻¹) | M_{w}/Mₙ |
| V88 | 110 | 4.5 | 5 | 60 | 10,600 | 8.34 |

In order to understand better the process, the polymerisation reaction as carried out under the above conditions was monitored by taking samples every hour.

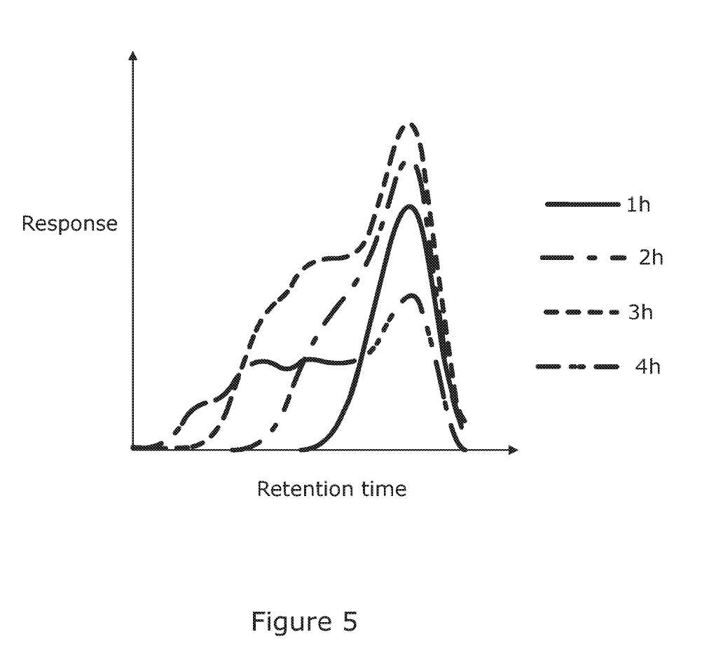

FIG. 5 is a GPC trace that shows the results, setting out the polymerisation of the carvone-based acrylate derivative at different reaction times. As conversion increases and the polymer chains grow, the level of branching also increases. This process of gradual branching is clearly observed in the GPC traces.

TABLE 12

| t (h) | Conv (%)^{a} | Mₙ^{b} | M_{w}/Mₙ^{b} |
|---|---|---|---|
| 1 | 32 | 4,600 | 1.70 |
| 2 | 42 | 6,500 | 2.84 |
| 3 | 49 | 8,600 | 4.82 |
| 4 | 60 | 10,600 | 8.34 |

| | |
|---|---|
| 5 | Crosslinked |

^{a}Determined by 1H NMR.
^{b}Determined by GPC-SEC in THF using PMMA standards.

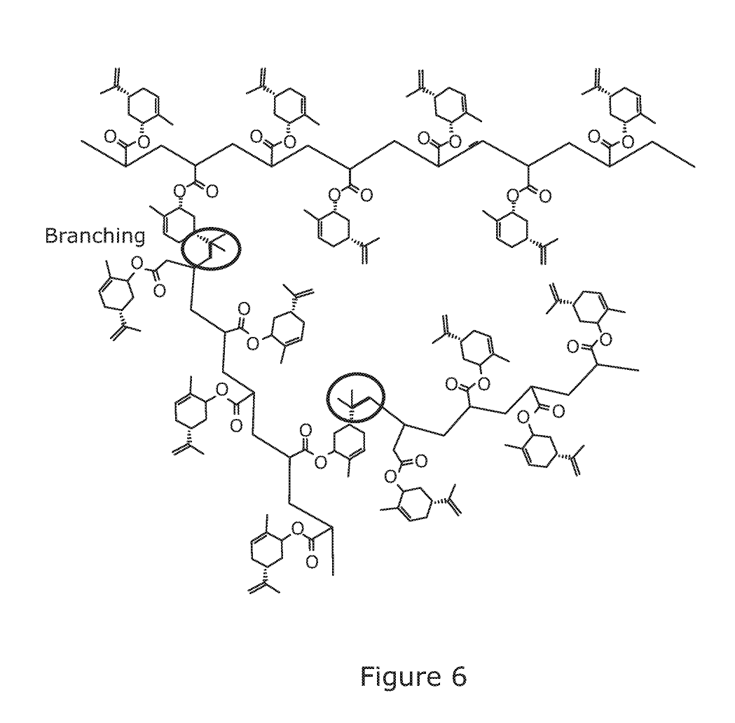

The proposed chemical structure of the polymer obtained from carvone-based acrylate is shown in FIG. 6. It can be seen that there is, therefore, a branched polymer obtained from the carvone-based acrylate monomer.

Thus soluble branched polymers can be synthesised from the carvone-based acrylate monomer.

These polymers are potentially useful materials for coating applications.

### Example 2c

### Molecular Weight Control

The molecular weight of a polymer is important because it determines many physical properties, such as transition temperatures, stiffness, strength and viscosity. Therefore it is crucial to be able to control the molecular weight.

In order to provide control over the polymerisation reactions based on the terpene/terpenoid monomers of the invention, a conventional CTA (DDM) was used.

Different concentrations of DDM were tested, and new terpene/terpenoid-based polymers with different molecular weights were successfully synthesised. The glass transition temperatures (T_{g}s) of the new polymers were measured and the differences were carefully studied.

Polymerisation reactions were carried out by mixing of 1 g of monomer, 0.5% wt of AIBN and DDM in 1.5 mL of cyclohexanone in a 10 mL round-bottomed flask. The mixture was previously degassed by freeze-pump-thaw technique and then heated up to 65° C. for 24 h. The Conversion % was determined by ¹H NMR. The Mₙ (g mol⁻¹) and the M_{w}/Mₙ were determined by GPC-SEC in THF using PMMA standards.

Results are set out in the Table 13 below. Many of these are confirmation of the results shown in the section “Initial discussion of the experimental work and the conclusions that can be drawn”

TABLE 13

| | Polymer | [DDM] | Conversion | Mₙ | M_{w}/ | Tg |
|---|---|---|---|---|---|---|
| Monomer | structure | % wt | (%) | (g mo1⁻¹) | Mₙ | (° C.) |
| 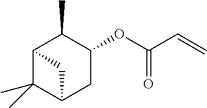 | 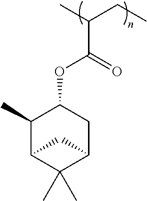 | 5 1 0.5 | 95 97 99 | 4,800 15,100 23,600 | 1.34 2.04 2.20 | 12 63 71 |
| 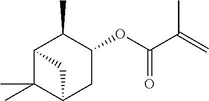 [SMILES: C=C(C)C(=O)O[C@@H]1C[C@@H]2C[C@H]([C@H]1C)C2(C)C] | 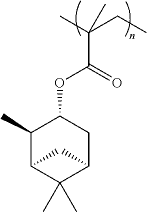 | 5 1 0.5 | 99 99 99 | 7,900 17,400 22,200 | 1.41 1.71 1.85 | 85 140 142 |
| 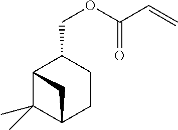 | 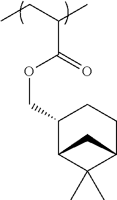 | 5 1 0.5 | 94% 98 97 | 4,200 27,100 31,500 | 1.31 1.63 2.06 | −8 40 41 |
| 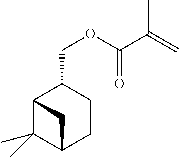 [SMILES: C=C(C)C(=O)OC[C@@H]1CC[C@H]2C[C@@H]1C2(C)C] | 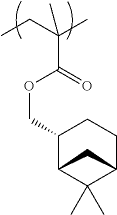 | 5 1 0.5 | 99 99 99 | 8,000 14,500 21,000 | 1.34 1.45 1.53 | 65 101 115 |
| 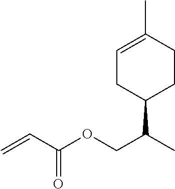 | 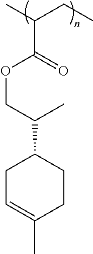 | 5 1 0.5 | 93 90 88 | 5,100 13,000 17,000 | 2.35 1.87 2.23 | −18 −10 −5 |
| 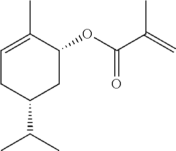 [SMILES: C=C(C)C(=O)O[C@@H]1C[C@H](C(C)C)CC=C1C] | 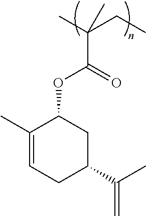 | 5 1 0.5 | 98 99 99 | 9,400 26,300 26,500 | 1.59 2.40 2.24 | 90 117 117 |

It can therefore be appreciated that a very wide range of T_{g}s has been observed. This flexibility should allow the synthesis of polymers with target physical properties.

### Example 2d

### Control Transfer Agents

To move towards a “greener” route, natural terpenes were tested as controlling agents in the polymerisation of the new terpene-based monomers. The use of terpenes as controlling agents is very attractive because they are naturally abundant and cheap and they provide a sustainable alternative.

All reactions were carried out by mixing of 1 g of monomer, 0.5% wt of AIBN, 1% of DDM and 1.5 mL of cyclohexanone in a 10 mL round-bottomed flask. The mixture was previously degassed by freeze-pump-thaw technique and then heated up to 65° C. for 24 h. The Conversion % was determined by ¹H NMR. The Mₙ (g mol⁻¹) and the M_{w}/Mₙ were determined by GPC-SEC in THF using PMMA standards.

Results are set out in the Table 14 below. Many of these are confirmation of the results shown in the section “Initial discussion of the experimental work and the conclusions that can be drawn”

TABLE 14

| | Polymer | Terpene | Conversion | Mₙ | M_{w}/ |
|---|---|---|---|---|---|
| Monomer | structure | (1%) | (%)^{a} | (g mol⁻¹)^{b} | Mₙ^{b} |
| 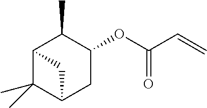 | 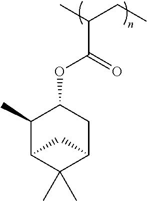 | γ-Terpinene Terpinolene | 64 45 | 35,000 22,600 | 1.30 1.46 |
| 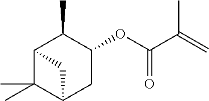 [SMILES: C=C(C)C(=O)O[C@@H]1C[C@@H]2C[C@H]([C@H]1C)C2(C)C] | 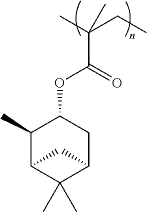 | γ-Terpinene Terpinolene | 65 44 | 46,700 50,000 | 3.22 3.88 |
| 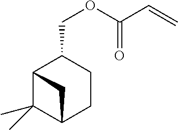 | 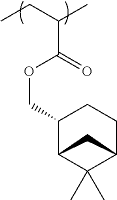 | γ-Terpinene Terpinolene | 67 67 | 21,300 17,100 | 1.45 1.62 |
| 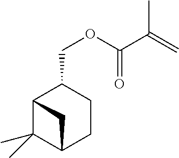 [SMILES: C=C(C)C(=O)OC[C@@H]1CC[C@H]2C[C@@H]1C2(C)C] | 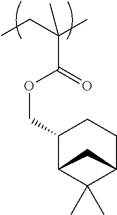 | γ-Terpinene Terpinolene | 84 78 | 38,000 28,200 | 1.92 1.97 |
| 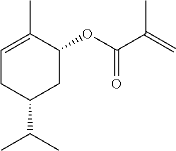 [SMILES: C=C(C)C(=O)O[C@@H]1C[C@H](C(C)C)CC=C1C] | 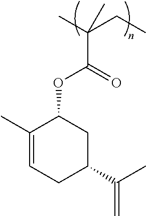 | γ-Terpinene Terpinolene | 78 65 | 45,100 37,500 | 2.59 1.83 |

It can therefore be seen that terpenes successfully provided controlled molecular weights in every case. If desired, longer reaction times could be used to increase conversion. Another alternative is the use of higher temperatures to increase the propagation rate of the monomers.

### CONCLUSIONS

In conclusion, new terpene/terpenoid-based polymers have been synthesised from the commercially available terpenes and terpenoids with good yields. The new polymers show a very wide range of T_{g} values.

It can therefore be appreciated that this flexibility should allow the synthesis of polymers with target physical properties.

Conventional chain transfer agents can be used to effectively control the polymerisation of the new terpene/terpenoid derivatives, such as acrylate and methacrylate derivatives. Natural terpenes, terpinene and terpinolene, have been proved to act as controlling agents in the polymerisation of the new monomers, with results comparable to those obtained with DDM, providing a green alternative.

1. A method for producing functionalised monomers, the method comprising:
a) forming a derivative of a terpene or terpenoid by incorporation of a hydroxyl group; and
b) esterifying the hydroxyl group of the derivative to introduce a moiety containing a vinyl group, so as to produce a functionalised monomer.

2. (canceled)

3. A method for producing functionalised monomers, the method comprising:
forming a derivative of a terpene or terpenoid by incorporating an organic group including a polymerisable functionality by catalytic introduction of a moiety containing a vinyl group, so as to produce a functionalised monomer.

4.-12. (canceled)

13. The method of claim 1, wherein the terpene or terpenoid is selected from the group consisting of: α-pinene, β-pinene, 3-carene, myrcene, terpinolene, limonene, α-terpinene, γ-terpinene, camphene, β-phellandrene, sabinene, p-cymene, ocimene, α-thujene, linalool, citronellal, thymol, carvacrol, verbenone, carvone, camphor, borneol, menthol, and terpineol.

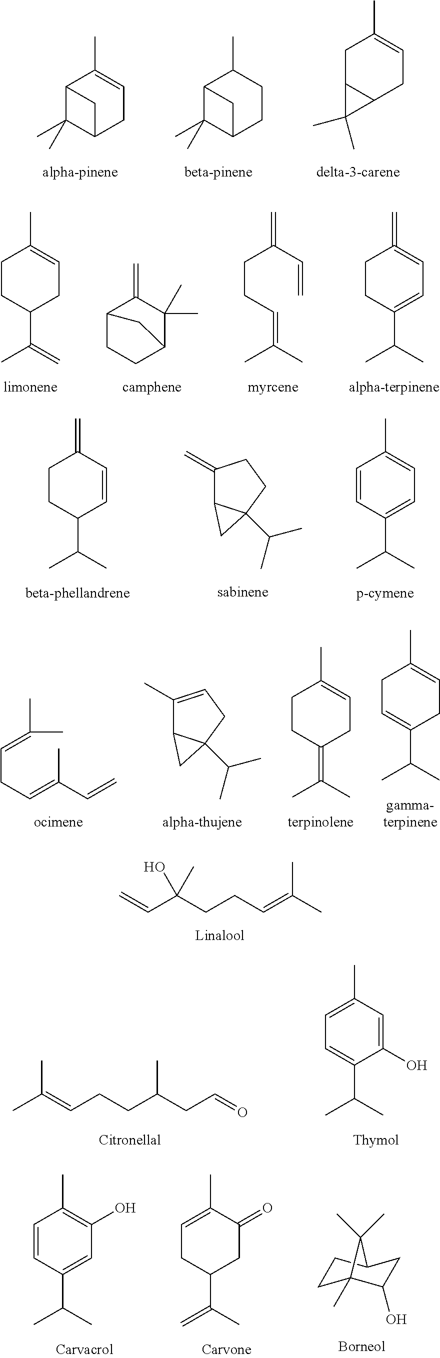

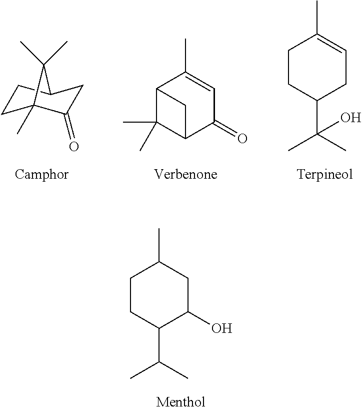

14. The method of claim 13, wherein the terpene or terpenoid is selected from the group consisting of α-pinene, β-pinene, 3-carene, myrcene, terpinolene, limonene, α-terpinene, γ-terpinene, camphene, thymol, carvacrol, verbenone, carvone, camphor, borneol, menthol, and terpineol.

15. The method of claim 14, wherein the terpene or terpenoid is selected from the group consisting of: α-pinene, β-pinene, terpinolene, limonene, camphene, camphor, carvone, verbenone, menthol, and terpineol.

16. The method of claim 1, wherein the monomer is a terpene or terpenoid that has been modified, wherein the terpene or terpenoid retains its C10 structural unit and the modification comprises the incorporation of a C1-C6 organic group, said organic group including a moiety containing a vinyl group as a polymerisable functionality, and said organic group being linked to the C10 structural unit by an ester linkage.

17. The method of claim 16, wherein the organic group is a C1-C4 organic group containing a vinyl group as a polymerisable functionality.

18. The method of claim 17, wherein the functionalised monomer is a terpene or terpenoid that has been modified, wherein the terpene or terpenoid retains its C10 structural unit and wherein the modification comprises the incorporation of an acrylate or methacrylate group.

19. The method of claim 1, wherein the terpene is carvone.

20. The method of claim 1, wherein the functionalised monomer is carvone acrylate.

21. The method of claim 3, wherein the terpene or terpenoid is selected from the group consisting of: α-pinene, β-pinene, 3-carene, myrcene, terpinolene, limonene, α-terpinene, γ-terpinene, camphene, β-phellandrene, sabinene, p-cymene, ocimene, α-thujene, linalool, citronellal, thymol, carvacrol, verbenone, carvone, camphor, borneol, menthol, and terpineol.

22. The method of claim 3, wherein the terpene or terpenoid is selected from the group consisting of α-pinene, β-pinene, 3-carene, myrcene, terpinolene, limonene, α-terpinene, γ-terpinene, camphene, thymol, carvacrol, verbenone, carvone, camphor, borneol, menthol, and terpineol.

23. The method of claim 22, wherein the terpene or terpenoid is selected from the group consisting of: α-pinene, β-pinene, terpinolene, limonene, camphene, camphor, carvone, verbenone, menthol, and terpineol.

24. The method of claim 3, wherein the monomer is a terpene or terpenoid that has been modified, wherein the terpene or terpenoid retains its C10 structural unit and the modification comprises the incorporation of a C1-C6 organic group, said organic group including a moiety containing a vinyl group as a polymerisable functionality, and said organic group being linked to the C10 structural unit by an ester linkage.

25. The method of claim 24, wherein the organic group is a C1-C4 organic group containing a vinyl group as a polymerisable functionality.

26. The method of claim 25, wherein the functionalised monomer is a terpene or terpenoid that has been modified, wherein the terpene or terpenoid retains its C10 structural unit and wherein the modification comprises the incorporation of an acrylate or methacrylate group.

27. The method of claim 3, wherein the terpene is α-pinene or β-pinene.

28. The method of claim 3, wherein the functionalised monomer is selected from the group consisting of: α-pinene acrylate, β-pinene acrylate, α-pinene methacrylate, and β-pinene methacrylate.

29. The method of claim 3, wherein the catalytic reaction is carried out with a carboxylic acid-containing monomer in the presence of a catalyst.

30. The method of claim 29, wherein the catalyst is a Pd(II)-catalyst.
